(19) United States
(12) Patent Application Publication
(10) Pub. No.: US 2025/0049656 A1
(43) Pub. Date: Feb. 13, 2025

(54) COMPOSITION FOR LIGHTENING KERATIN FIBRES AND PROCESS FOR LIGHTENING KERATIN FIBRES USING THIS COMPOSITION

(71) Applicant: L'OREAL, Paris (FR)

(72) Inventors: Stéphane BLAIS, AULNAY-SOUS-BOIS (FR); Stéphane SABELLE, AULNAY-SOUS-BOIS (FR); Nawel BAGHDADLI, AULNAY-SOUS-BOIS (FR)

(21) Appl. No.: 18/719,686

(22) PCT Filed: Dec. 13, 2022
(86) PCT No.: PCT/EP2022/085724

§ 371 Date: Jun. 13, 2024

(30) Foreign Application Priority Data
Dec. 16, 2021 (FR) FR2113731

(51) Int. Cl. A61K 8/22; A61K 8/25; A61K 8/37; A61K 8/49; A61Q 5/08; A61Q 5/10

(52) U.S. Cl. CPC A61K 8/22; A61K 8/25; A61K 8/37; A61K 8/375; A61K 8/4973; A61Q 5/08; A61Q 5/10; A61K 2800/88

(57) ABSTRACT

The present invention relates to a composition for lightening keratin fibres, comprising at least one chemical oxidizing agent, at least one (bi)carbonate, at least one silicate and at least one compound chosen from esters and/or amides and/or imines, and to a process for lightening keratin fibres using this composition.

### TECHNICAL FIELD OF THE INVENTION

The present invention relates to a composition for lightening keratin fibres, comprising at least one chemical oxidizing agent, at least one (bi)carbonate, at least one silicate and at least one compound chosen from esters and/or amides and/or imines, and to a process for lightening keratin fibres using this composition.

### BACKGROUND OF THE INVENTION

When a person wishes to change hair colour, in particular when he or she wishes to obtain a lighter colour than his or her original colour, it is often necessary to carry out, beforehand, hair lightening or bleaching. To do this, lightening or bleaching products are used.

The lightening of hair is evaluated by the “tone depth”, which characterizes the degree or level of lightening. The notion of “tone” is based on the classification of natural shades, one tone separating each shade from the shade immediately following or preceding it. This definition and the classification of natural shades are well known to hairstyling professionals and are published in the book Sciences des traitements capillaires [The Science of Hair Care] by Charles Zviak, 1988, published by Masson, pp. 215 and 278. The tone depths range from 1 (black) to 10 (very light blond), one unit corresponding to one tone; the higher the figure, the lighter the shade.

It is known practice to lighten or bleach the hair with lightening or bleaching compositions containing at least one chemical oxidizing agent, under alkaline pH conditions in the vast majority of cases. The role of this oxidizing agent is to degrade the melanin of the hair, which, depending on the nature of the oxidizing agent present and on the pH conditions, leads to more or less pronounced lightening of the fibres. Thus, for relatively mild lightening, the oxidizing agent is generally hydrogen peroxide. When greater lightening is desired, in particular when the treated hair is dark, persulfates are normally used, in the presence of hydrogen peroxide. However, the lightening obtained through the action of such a combination is not always satisfactory since hair with fairly unattractive orangey-yellow shades that are very different from natural shades is obtained, thereby complicating the subsequent colouring by limiting it to the obtaining of warm tones. Furthermore, persulfate-based lightening compositions can lead to a degradation of the quality of the fibre. In addition, the persulfate-based compositions are generally not chemically compatible with the presence of oxidation dyes and/or direct dyes in order to be able to bleach the hair fibre and dye it in a single step. Thus, when it is desired to simultaneously bleach and dye keratin fibres, a two-step process is used with a first step of bleaching the keratin fibres followed by a second step of dyeing the keratin fibres using a dye composition comprising one or more direct dyes and/or one or more oxidation dyes.

There is thus a real need to develop a composition which makes it possible to obtain efficient lightening of keratin fibres, in particular of dark keratin fibres, with a less yellow and more natural result. Such a composition will also have to be more attentive to the quality of the fibres, minimizing in particular their degradation. Lastly, such a composition should also be compatible with the presence of direct dyes and/or oxidation dyes in order to obtain a good colour build-up, intense and chromatic colours and also to enable a broader range of colours to be achieved.

The applicant has discovered, surprisingly, that all or some of these objectives can be achieved by the composition according to the present invention.

### SUMMARY OF THE INVENTION

According to a first aspect, a subject of the present invention is a composition comprising:
i) one or more chemical oxidizing agents chosen from hydrogen peroxide, hydrogen peroxide-generating systems other than peroxygenated salts, and mixtures thereof;
ii) one or more compounds chosen from carbonates, carbonate-generating systems, bicarbonates, bicarbonate-generating systems, and mixtures thereof, preferably from carbonates, bicarbonates, and mixtures thereof;
iii) one or more silicates;
iv) one or more compounds chosen from esters, amides, imines, and mixtures thereof; in which:
   the silicate(s) are present in the composition in a total content ranging from 1% to 40% by weight relative to the total weight of the composition; and
   the composition comprises a total content of persulfates of less than 5% by weight relative to the total weight of the composition.

According to a second aspect, a subject of the present invention is a process for lightening keratin fibres, comprising the application to the keratin fibres of a composition as defined above.

According to a third aspect, a subject of the present invention is a process for simultaneously bleaching and dyeing keratin fibres, comprising the application to the keratin fibres of a composition comprising ingredients i) to iv) as defined above and v) one or more colouring agents chosen from direct dyes, oxidation dyes, and mixtures thereof.

According to a fourth aspect, a subject of the present invention is the use of a composition comprising ingredients i) to iv) as defined above for lightening keratin fibres, preferably for lightening keratin fibres while at the same time making them less yellow.

According to a fifth aspect, a subject of the present invention is the use of a composition comprising ingredients i) to iv) as defined above and v) one or more colouring agents chosen from direct dyes, oxidation dyes, and mixtures thereof, for the simultaneous bleaching and dyeing of keratin fibres.

According to a sixth aspect, a subject of the present invention is a device having multiple separate compartments (kit), comprising:
a first compartment containing a composition (A) comprising:
   one or more chemical oxidizing agents chosen from hydrogen peroxide, hydrogen peroxide-generating systems other than peroxygenated salts, and mixtures thereof, preferably hydrogen peroxide; and
a second compartment containing a composition (B) comprising:
   one or more compounds chosen from carbonates, carbonate-generating systems, bicarbonates, bicarbonate-generating systems, and mixtures thereof, preferably chosen from carbonates, bicarbonates, and mixtures thereof; and
   one or more silicates; and
   optionally one or more colouring agents chosen from direct dyes, oxidation dyes, and mixtures thereof; and
   optionally one or more compounds chosen from esters, amides, imines, and mixtures thereof; and
optionally a third compartment containing a composition (C) comprising:
   one or more colouring agents chosen from direct dyes, oxidation dyes, and mixtures thereof; and
   optionally one or more compounds chosen from esters, amides, imines, and mixtures thereof;
      it being understood that composition (B) or at least one of compositions (B) and (C), when composition (C) is present, comprises one or more compounds chosen from esters, amides, imines, and mixtures thereof.

### BRIEF DESCRIPTION OF THE FIGURES

FIG. 1 is a graph representing the variation in intensity L* as a function of the parameter b* for the compositions according to the invention (Example 2) and the comparative compositions based on persulfate (Example 1), the values of L* and b* being measured in the CIE L*a*b* system.

### DETAILED DESCRIPTION OF THE INVENTION

For the purposes of the present invention, and unless otherwise indicated:
the term “keratin fibres” means fibres of human or animal origin, such as head hair, bodily hair, the eyelashes, the eyebrows, wool, angora, cashmere or fur. According to the present invention, the keratin fibres are preferably human keratin fibres, more preferentially the hair.
the term “ester” means a compound comprising at least one ester function.
the term “amide” means a compound comprising at least one amide function.
the term “imine” means a compound comprising at least one imine function.
the term “salt” means an addition salt with a mineral or organic acid or base. The addition salts with an acid are chosen in particular from addition salts with acids such as hydrochloric acid, hydrobromic acid, sulfuric acid, citric acid, succinic acid, tartaric acid, lactic acid, tosylic acid, benzenesulfonic acid, phosphoric acid or acetic acid. The addition salts with a base are chosen in particular from addition salts with bases such as the basifying agents defined below, in particular alkali metal hydroxides, alkaline earth metal hydroxides, aqueous ammonia, amines or alkanolamines.
the term “alkyl group” means a linear or branched, saturated hydrocarbon radical comprising from 1 to 30 carbon atoms, preferentially from 1 to 26 carbon atoms, more preferentially from 1 to 22 carbon atoms, for example methyl, ethyl, n-propyl, isopropyl, butyl, n-pentyl, n-hexyl, n-decyl, n-dodecyl, tetradecyl, hexadecyl or eicosyl.
the term “(Cₓ-C_{y})alkyl group” means an alkyl group comprising from x to y carbon atoms.
the term “alkenyl group” means a linear or branched hydrocarbon radical comprising one or more conjugated or nonconjugated ethylenic unsaturations and from 1 to 30 carbon atoms, preferably from 1 to 26 carbon atoms, more preferentially from 1 to 22 carbon atoms, for example ethylenyl, n-propylenyl, isopropylenyl, butylenyl, n-pentylenyl, n-hexylenyl, n-decylenyl, n-dodecylenyl, tetradecylenyl, hexadecylenyl or eicosylenyl.
the term “(Cₓ-C_{y})alkenyl” group means an alkenyl group comprising from x to y carbon atoms.
the term “(poly)(hydroxy)alkyl group” means an alkyl group or a hydroxyalkyl group or a polyhydroxyalkyl group.
the term “hydroxyalkyl group” means an alkyl group substituted by a hydroxyl group (—OH);
the term “polyhydroxyalkyl group” means an alkyl group substituted by at least two hydroxyl groups (—OH).
the term “(hetero)cyclic group” means a cyclic or heterocyclic group.
the term “cyclic group” means a mono- or polycyclic, fused or nonfused, saturated or unsaturated, in particular aromatic, carbocycle comprising from 6 to 22 carbon atoms, it being possible for said cyclic group to be substituted by one or more identical or different groups, in particular chosen from: (C₁-C₆) alkyl, (C₁-C₆) alkoxy, (C₁-C₆) hydroxyalkyl, hydroxyl (—OH) or carboxyl (—COOH).
the term “heterocyclic group” means a mono- or polycyclic, fused or nonfused, saturated or unsaturated, in particular aromatic, group comprising from 5 to 22 ring members and containing from 1 to 3 heteroatoms chosen from nitrogen, oxygen and sulfur atoms, it being possible for said heterocyclic group to be substituted by one or more identical or different groups, in particular chosen from: (C₁-C₆) alkyl, (C₁-C₆) alkoxy, (C₁-C₆) hydroxyalkyl, hydroxyl (—OH) or carboxyl (—COOH).
the term “silicate” means a silicic acid salt.
the term “colouring agent” means an oxidation dye, a direct dye or a pigment.
the term “oxidation dye” means an oxidation dye precursor chosen from oxidation bases and couplers. Oxidation bases and couplers are colourless or sparingly coloured compounds, which, via a condensation reaction in the presence of an oxidizing agent, give a coloured species.
the term “direct dye” means a natural and/or synthetic dye, including in the form of an extract or extracts, other than oxidation dyes. These are coloured compounds that will spread superficially on the fibre. They may be ionic or nonionic, i.e. anionic, cationic, neutral or nonionic.
the term “chemical oxidizing agent” means an oxidizing agent other than atmospheric oxygen.

Unless otherwise indicated, when compounds are mentioned in the present patent application, this also includes the optical isomers thereof, the geometrical isomers thereof, the tautomers thereof, the salts thereof or the solvates thereof such as hydrates, and mixtures thereof.

The terms “at least one” and “one or more” are synonymous and may be used interchangeably.

The terms “lightening” and “bleaching” are synonymous and may be used interchangeably.

### Composition

According to a first aspect, a subject of the present invention is a composition comprising ingredients i) to iv) as defined above.

The applicant has noted, surprisingly, that the composition according to the present invention makes it possible to obtain efficient lightening of keratin fibres with a less yellow and more natural result. When the colour of the keratin fibres treated by the composition according to the invention is compared to the colour of the keratin fibres treated by lightening compositions known from the prior art, values of b*, measured in the CIE L* a* b* system, are observed that are lower for the composition according to the invention than for the lightening compositions known from the prior art, at equivalent intensity level L*.

Moreover, the composition according to the invention is more attentive to the quality of the fibres, minimizing in particular their degradation.

According to a preferred embodiment, the composition according to the invention comprises:
i) hydrogen peroxide;
ii) one or more compounds chosen from carbonates, bicarbonates, and mixtures thereof;
iii) one or more silicates;
iv) one or more compounds chosen from esters, amides, imines, and mixtures thereof; it being understood that:
   the silicate(s) are present in the composition in a total content ranging from 1% to 40% by weight relative to the total weight of the composition; and
   the composition comprises a total content of persulfates of less than 5% by weight relative to the total weight of the composition.

### Chemical Oxidizing Agents

The composition according to the invention comprises i) one or more chemical oxidizing agents chosen from hydrogen peroxide, hydrogen peroxide-generating systems other than peroxygenated salts, and mixtures thereof.

The hydrogen peroxide-generating systems other than peroxygenated salts may be chosen from urea peroxide, polymeric complexes that can release hydrogen peroxide, oxidases, and mixtures thereof.

As examples of polymeric complexes that can release hydrogen peroxide, mention may be made of polyvinylpyrrolidone/H₂O₂ in particular in powder form, and the other polymeric complexes described in U.S. Pat. Nos. 5,008,093, 3,376,110 and 5,183,901.

Oxidases can produce hydrogen peroxide in the presence of a suitable substrate, for instance glucose in the case of glucose oxidase or uric acid with uricase.

According to a particular embodiment, the hydrogen peroxide and/or the hydrogen peroxide-generating system(s) other than peroxygenated salts may be added to the composition according to the invention just before it is applied to the keratin fibres. The intermediate composition(s) comprising the hydrogen peroxide and/or the hydrogen peroxide-generating system(s) other than peroxygenated salts can be referred to as oxidizing compositions and can also contain various additional compounds or various adjuvants conventionally used in compositions for lightening keratin fibres.

According to a preferred embodiment, the composition according to the invention comprises hydrogen peroxide as chemical oxidizing agent.

The chemical oxidizing agent(s) are preferably present in the composition in a total content ranging from 1% to 12% by weight, more preferentially ranging from 3% to 9% by weight, even more preferentially ranging from 3.5% to 8.5% by weight, relative to the total weight of the composition.

According to a preferred embodiment, the hydrogen peroxide is present in the composition in a total content ranging from 1% to 12% by weight, preferably ranging from 3% to 9% by weight, more preferentially ranging from 3.5% to 8.5% by weight, relative to the total weight of the composition.

(Bi)Carbonates and/or (Bi)Carbonate-Generating Systems

The composition according to the invention additionally comprises ii) one or more compounds chosen from carbonates, carbonate-generating systems, bicarbonates, bicarbonate-generating systems, and mixtures thereof.

According to a preferred embodiment, the composition according to the invention additionally comprises ii) one or more compounds chosen from carbonates, bicarbonates, and mixtures thereof.

According to a more preferred embodiment, the composition according to the invention additionally comprises ii) one or more compounds chosen from ammonium carbonate, ammonium bicarbonate, and mixtures thereof.

The compound(s) ii) are present in the composition preferably in a total content ranging from 0.01% to 20% by weight, preferably ranging from 1% to 15% by weight, more preferentially ranging from 2% to 15% by weight, even more preferentially ranging from 4% to 15% by weight, relative to the total weight of the composition.

Carbonates and/or Carbonate-Generating Systems

The term “carbonate-generating system” means a system which generates carbonate in situ, for instance carbon dioxide in water or percarbonate in water.

Preferably, the carbonate(s) are chosen from:
alkali metal carbonates;
alkaline earth metal carbonates;
lanthanide carbonates;
transition metal carbonates;
bismuth carbonate;
cadmium carbonate;
thallium carbonate;
zinc carbonate;
the compounds of formula (N⁺R¹R²R³R⁴)₂, —CO₃²⁻ in which R¹, R², R³ and R⁴ represent, independently of one another, a hydrogen atom or a (C₁-C₄)alkyl group optionally substituted by a hydroxyl group;
guanidine carbonate;
mixtures thereof.

More preferentially, the carbonate(s) are chosen from sodium carbonate, potassium carbonate, caesium carbonate, lithium carbonate, magnesium carbonate, calcium carbonate, barium carbonate, strontium carbonate, cerium carbonate, lanthanum carbonate, yttrium carbonate, copper(II) carbonate, manganese carbonate, nickel carbonate, silver carbonate, zirconium carbonate, bismuth carbonate, cadmium carbonate, thallium carbonate, zinc carbonate, ammonium carbonate, guanidine carbonate, tetraethylammonium carbonate, and mixtures thereof.

Even more preferentially, the carbonate(s) are chosen from sodium carbonate, potassium carbonate, caesium carbonate, magnesium carbonate, calcium carbonate, cerium carbonate, manganese carbonate, zinc carbonate, ammonium carbonate, guanidine carbonate, and mixtures thereof.

Most preferentially, the carbonate(s) are chosen from sodium carbonate, potassium carbonate, magnesium carbonate, calcium carbonate, ammonium carbonate, and mixtures thereof.

According to a particularly preferred embodiment, the carbonate included in the composition is ammonium carbonate.

The carbonate(s) and/or the carbonate-generating system(s) are preferably present in the composition in a total content ranging from 0.01% to 20% by weight, more preferentially ranging from 1% to 20% by weight, even more preferentially ranging from 1% to 10% by weight, relative to the total weight of the composition.

According to a preferred embodiment, the carbonate(s) are present in the composition in a total content ranging from 0.01% to 20% by weight, preferably ranging from 1% to 20% by weight, more preferentially ranging from 1% to 10% by weight, relative to the total weight of the composition.

According to a preferred embodiment, the compound(s) ii) are chosen from carbonates, carbonate-generating systems, and mixtures thereof, preferably from carbonates.

Bicarbonates and/or Bicarbonate-Generating Systems

The term “bicarbonate-generating system” means a system which generates bicarbonate in situ, for instance carbon dioxide in water or by buffering a carbonate with a mineral or organic acid.

Preferably, the bicarbonate(s) are chosen from:
alkali metal bicarbonates;
alkaline earth metal bicarbonates;
the compounds of formula N⁺R¹R²R³R⁴, —HCO₃⁻ in which R¹, R², R³ and R⁴ represent, independently of one another, a hydrogen atom or a (C₁-C₄)alkyl group optionally substituted by a hydroxyl group;
aminoguanidine bicarbonate;
mixtures thereof.

More preferentially, the bicarbonate(s) are chosen from sodium bicarbonate, potassium bicarbonate, lithium bicarbonate, caesium bicarbonate, calcium bicarbonate, magnesium bicarbonate, ammonium bicarbonate, choline bicarbonate, triethylammonium bicarbonate, aminoguanidine bicarbonate, and mixtures thereof.

Even more preferentially, the bicarbonate(s) are chosen from sodium bicarbonate, potassium bicarbonate, caesium bicarbonate, calcium bicarbonate, magnesium bicarbonate, ammonium bicarbonate, and mixtures thereof.

Most preferentially, the bicarbonate(s) are chosen from sodium bicarbonate, potassium bicarbonate, ammonium bicarbonate, and mixtures thereof.

According to a particularly preferred embodiment, the bicarbonate included in the composition is ammonium bicarbonate.

The bicarbonates may originate from a natural water, for example spring water from the Vichy basin or from La Roche Posay or Badoit water.

The bicarbonate(s) and/or the bicarbonate-generating system(s) are preferably present in the composition in a total content ranging from 0.01% to 20% by weight, more preferentially ranging from 1% to 15% by weight, even more preferentially ranging from 2% to 15% by weight, most preferentially ranging from 4% to 15% by weight, relative to the total weight of the composition.

According to a preferred embodiment, the bicarbonate(s) are present in a total content ranging from 0.01% to 20% by weight, preferably ranging from 1% to 15% by weight, more preferentially ranging from 2% to 15% by weight, even more preferentially ranging from 4% to 15% by weight, relative to the total weight of the composition.

According to a preferred embodiment, the compound(s) ii) are chosen from bicarbonates, bicarbonate-generating systems, and mixtures thereof, preferably from bicarbonates.

### Silicates

The composition according to the invention also comprises iii) one or more silicates.

The silicate(s) are preferably water-soluble.

The term “water-soluble silicate” means a silicate which has a solubility in water at ordinary room temperature (25° C.) and at atmospheric pressure (760 mmHg) of greater than 0.5% by weight, preferably greater than 1% by weight.

Preferably, the silicate(s) are chosen from alkali metal silicates, alkaline earth metal silicates, aluminium silicates, trimethylammonium silicates, and mixtures thereof.

More preferentially, the silicate(s) are chosen from sodium silicates, potassium silicates, calcium silicates, aluminium silicates, trimethylammonium silicates, and mixtures thereof.

Even more preferentially, the silicate(s) are chosen from sodium silicates.

Preferably, the silicate(s) are chosen from the compounds having the INCI name Sodium Silicate (CAS: [1344-09-8]) and/or Sodium Metasilicate (CAS: [6834-92-0]).

The silicate(s) are present in the composition in a total content ranging from 1% to 40% by weight, preferably ranging from 2% to 35% by weight, more preferentially ranging from 3% to 35% by weight, even more preferentially ranging from 4% to 20% by weight, relative to the total weight of the composition.

The weight ratio of total amount of carbonate(s) and/or carbonate-generating system(s)/total amount of silicate(s) is preferably from 0.00025 to 20, more preferentially from 0.028 to 10, even more preferentially from 0.028 to 3.4.

According to a preferred embodiment, the weight ratio of total amount of carbonate(s)/total amount of silicate(s) is from 0.00025 to 20, preferably from 0.028 to 10, more preferentially from 0.028 to 3.4.

The weight ratio of total amount of carbonate(s) and/or carbonate-generating system(s)/total amount of chemical oxidizing agent(s) is preferably from 0.0008 to 20, more preferentially from 0.1 to 6.6, even more preferentially from 0.1 to 2.9.

According to a preferred embodiment, the weight ratio of total amount of carbonate(s)/total amount of chemical oxidizing agent(s) is from 0.0008 to 20, preferably from 0.1 to 6.6, more preferentially from 0.1 to 2.9.

According to a preferred embodiment, the weight ratio of total amount of carbonate(s)/total amount of hydrogen peroxide is from 0.0008 to 20, preferably from 0.1 to 6.6, more preferentially from 0.1 to 2.9.

The weight ratio of total amount of bicarbonate(s) and/or bicarbonate-generating system(s)/total amount of silicate(s) is preferably from 0.00025 to 20, more preferentially from 0.02 to 7.5, even more preferentially from 0.05 to 5.

According to a preferred embodiment, the weight ratio of total amount of bicarbonate(s)/total amount of silicate(s) is from 0.00025 to 20, preferably from 0.02 to 7.5, more preferentially from 0.05 to 5.

The weight ratio of total amount of bicarbonate(s) and/or bicarbonate-generating system(s)/total amount of chemical oxidizing agent(s) is preferably from 0.0008 to 20, more preferentially from 0.11 to 5, even more preferentially from 0.2 to 4.2.

According to a preferred embodiment, the weight ratio of total amount of bicarbonate(s)/total amount of chemical oxidizing agent(s) is from 0.0008 to 20, preferably from 0.11 to 5, more preferentially from 0.2 to 4.2.

According to a more preferred embodiment, the weight ratio of total amount of bicarbonate(s)/total amount of hydrogen peroxide is from 0.0008 to 20, preferably from 0.11 to 5, more preferentially from 0.2 to 4.2.

The weight ratio of total amount of carbonate(s) and/or carbonate-generating system(s)/total amount of bicarbonate(s) and/or bicarbonate-generating system(s) is preferably from 0.0005 to 2000, more preferentially from 0.06 to 20, even more preferentially from 0.06 to 5.

According to a preferred embodiment, the weight ratio of total amount of carbonate(s)/total amount of bicarbonate(s) is from 0.0005 to 2000, preferably from 0.06 to 20, more preferentially from 0.06 to 5.

### Esters, Amides and Imines

The composition according to the invention additionally comprises iv) one or more compounds chosen from esters, amides, imines, and mixtures thereof.

Preferably, the compound(s) iv) are chosen from the compounds of formula (I) below, salts thereof, optical isomers thereof, geometric isomers thereof, tautomers thereof, solvates thereof, and mixtures thereof:

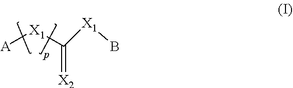

in which formula (I):
A represents a saturated or unsaturated, linear or branched hydrocarbon group comprising from 1 to 30 carbon atoms, optionally interrupted by one or more nonadjacent oxygen atoms and/or by one or more identical or different groups chosen from: —CO—, —OCO—, —COO—, and/or optionally substituted by one or more identical or different groups chosen from: a hydroxyl group (—OH), a group —COOAk1, a (hetero)cyclic group optionally substituted by one or more hydroxyl groups (—OH);
X₁ independently represents an oxygen atom or a group —NR—, preferably an oxygen atom;
X₂ represents an oxygen atom or a group —NR—, preferably an oxygen atom;
p=0 or 1;
B represents a saturated or unsaturated, linear or branched or cyclic hydrocarbon group, comprising from 1 to 30 carbon atoms, optionally substituted by one or more identical or different groups chosen from:
a (hetero)cyclic group optionally substituted by one or more identical or different groups chosen from: hydroxyl (—OH), —OR″, —COOA′, —OCOA′, (poly)(hydroxy)alkyl;
a group —OR′;
a group —COOA′;
a group —OCOA′;
   and/or optionally interrupted by one or more nonadjacent oxygen atoms and/or by one or more identical or different groups chosen from: —OCO—, —COO—;
A′ represents a saturated or unsaturated, linear or branched hydrocarbon group comprising from 1 to 30 carbon atoms, optionally interrupted by one or more nonadjacent oxygen atoms and/or by one or more identical or different groups chosen from: —CO—, —OCO—, —COO—, and/or optionally substituted by one or more identical or different groups chosen from: hydroxyl (—OH), —COOAk1;
R represents a hydrogen atom or an alkyl group, in particular a (C₁-C₄) alkyl group;
R′ represents a hydrogen atom or an alkyl group optionally substituted by one or more identical or different groups chosen from: hydroxyl (—OH) or —OCOA′;
R″ represents:
an alkyl group optionally substituted by one or more hydroxyl groups (—OH); or
a (hetero)cyclic group optionally substituted by one or more identical or different groups chosen from: hydroxyl (—OH), (poly)(hydroxy)alkyl;
Ak1 represents an alkyl group optionally substituted by —COOAk2;
Ak2 represents an alkyl group;
it being possible for R and B to form a heterocycle together with the nitrogen atom bearing them;
it being possible for A and B to form together a heterocycle optionally substituted in particular by one or more identical or different groups chosen from:
a (hetero)cyclic group optionally substituted in particular by one or more identical or different groups chosen from: hydroxyl (—OH), (poly)(hydroxy)alkyl;
a hydroxyl group (—OH);
a group —OR″;
a (poly)(hydroxy)alkyl group;
it being understood that if X₁ represents a group —NR— then A and B may represent a hydrogen atom.

More preferentially, the compound(s) iv) are chosen from the following compounds 1 to 93, salts thereof, optical isomers thereof, geometric isomers thereof, tautomers thereof, solvates thereof, and mixtures thereof:

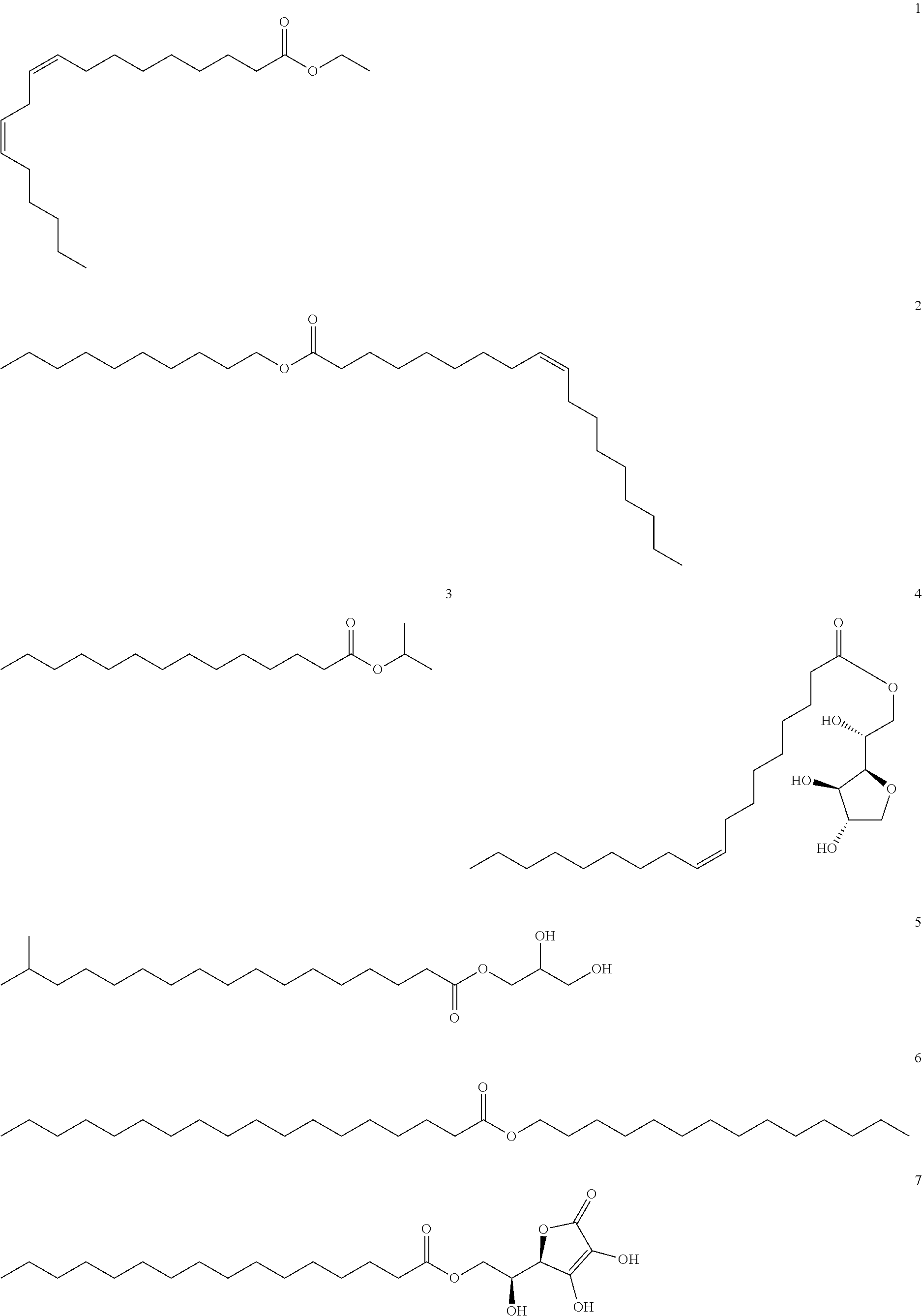

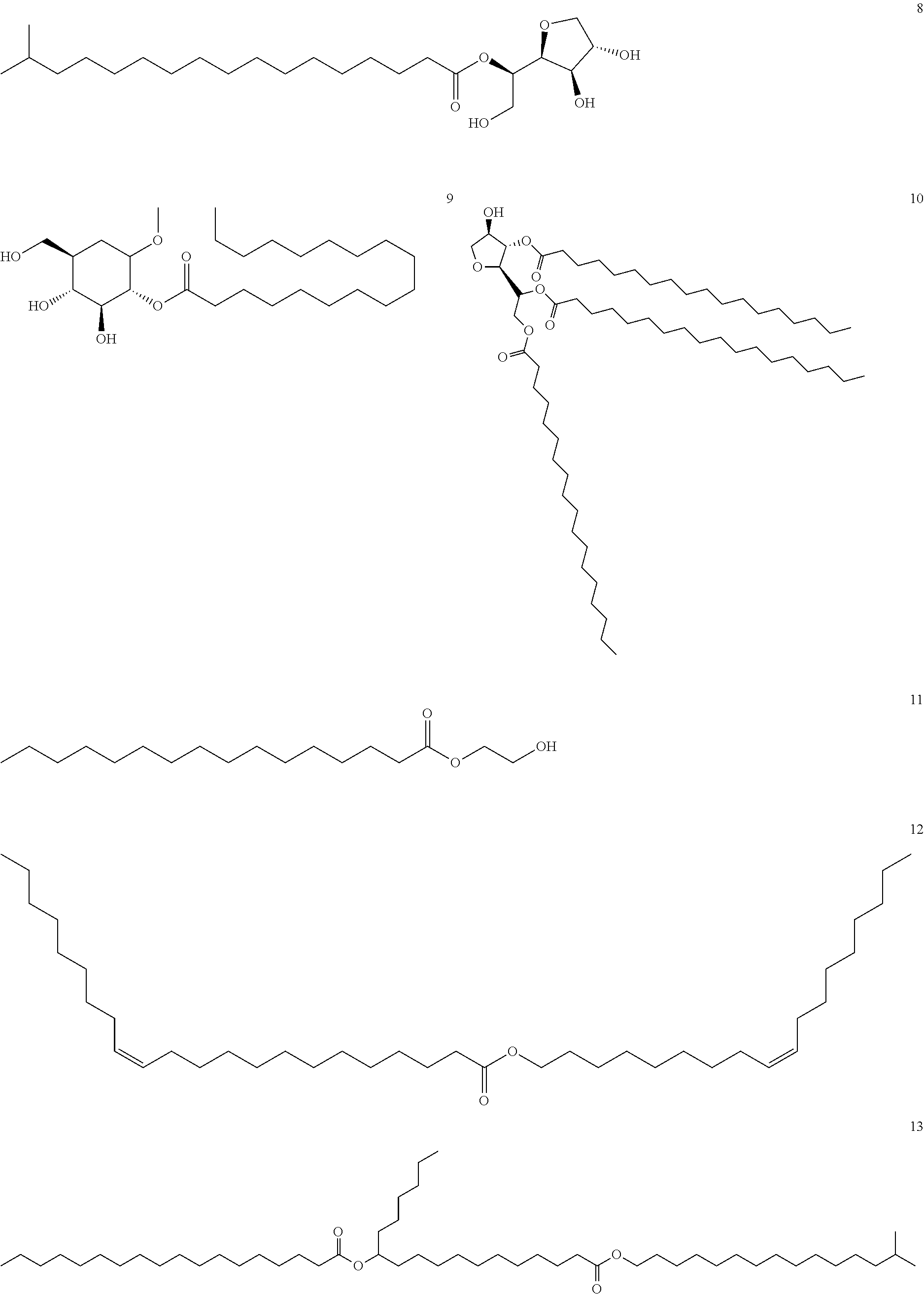

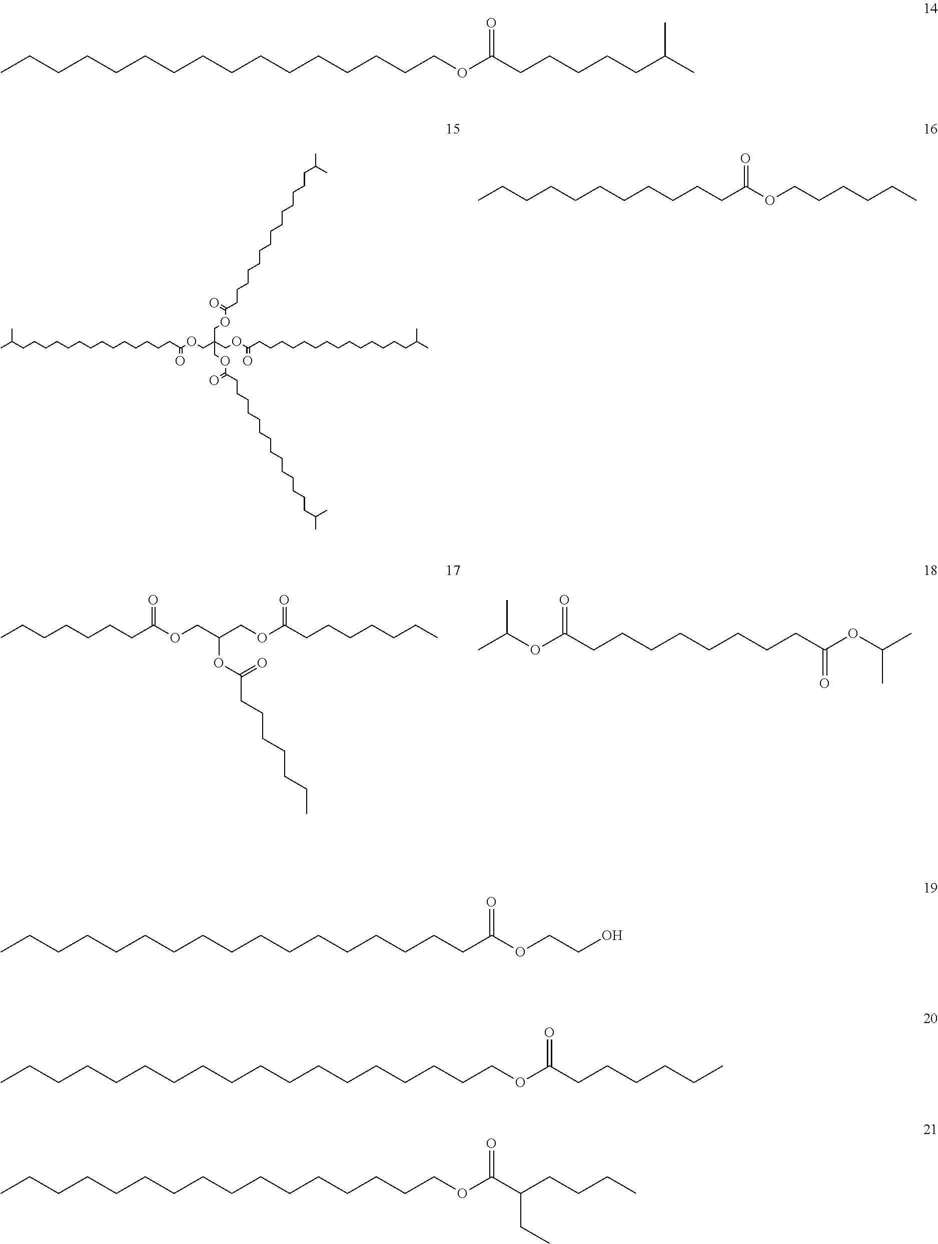

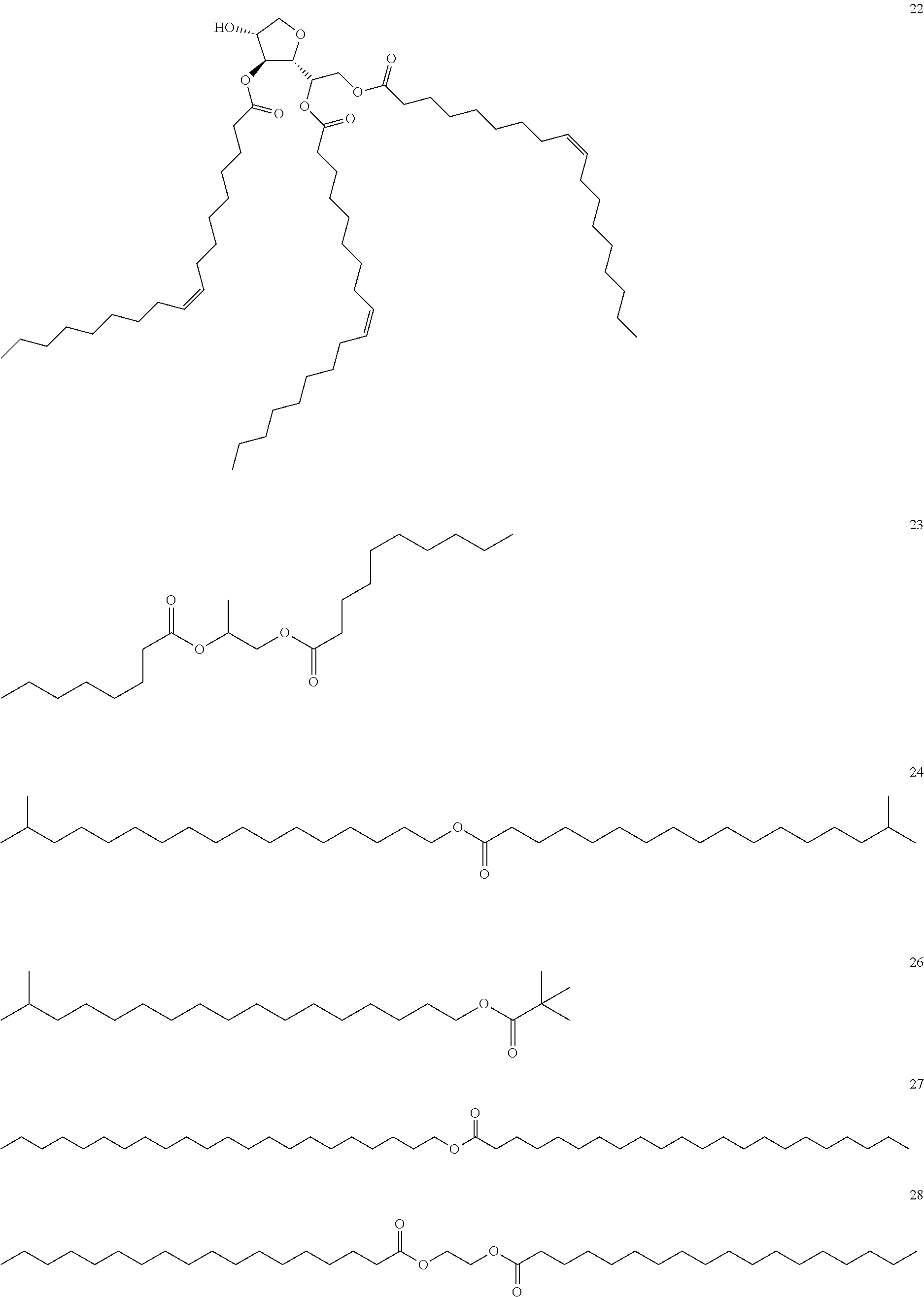

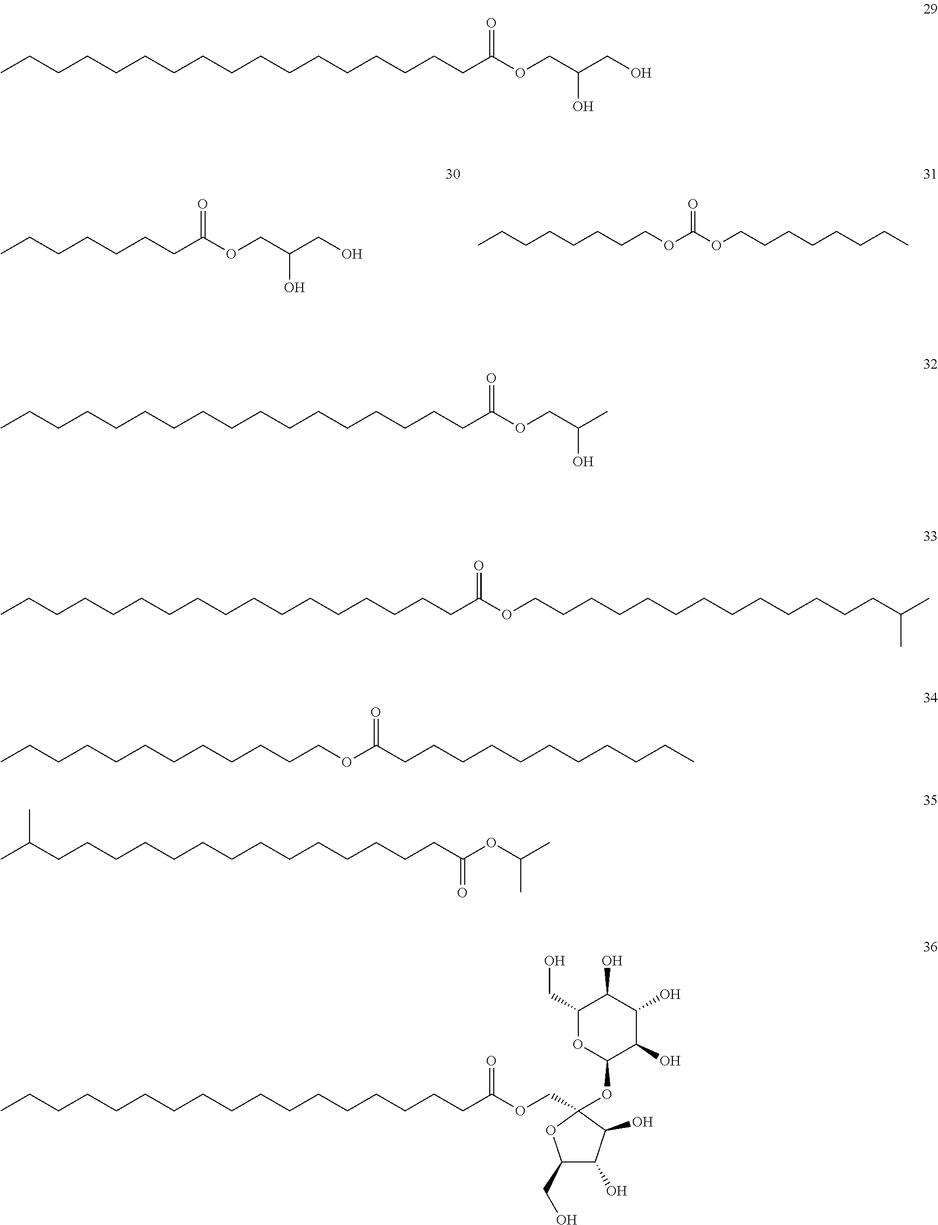

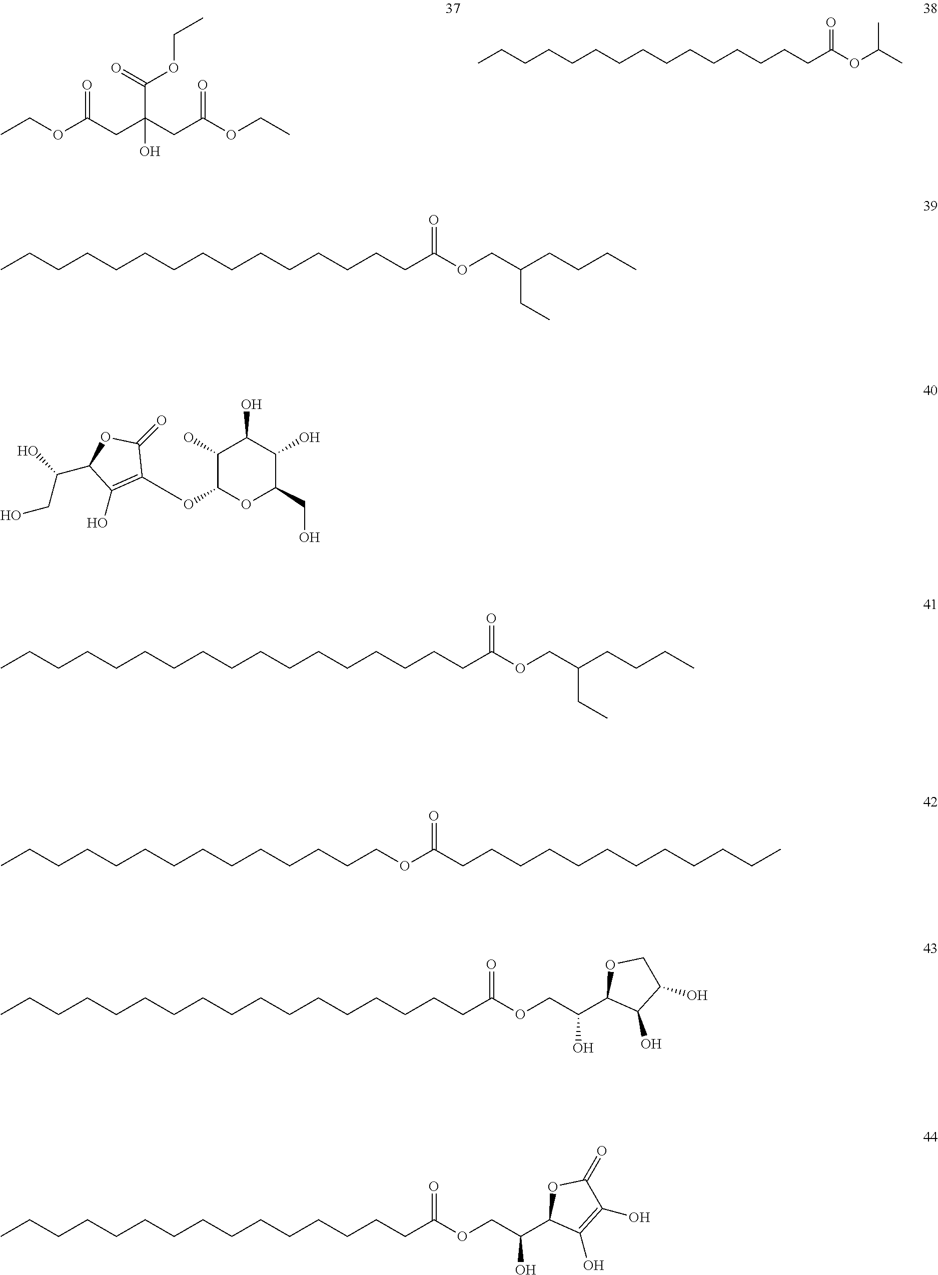

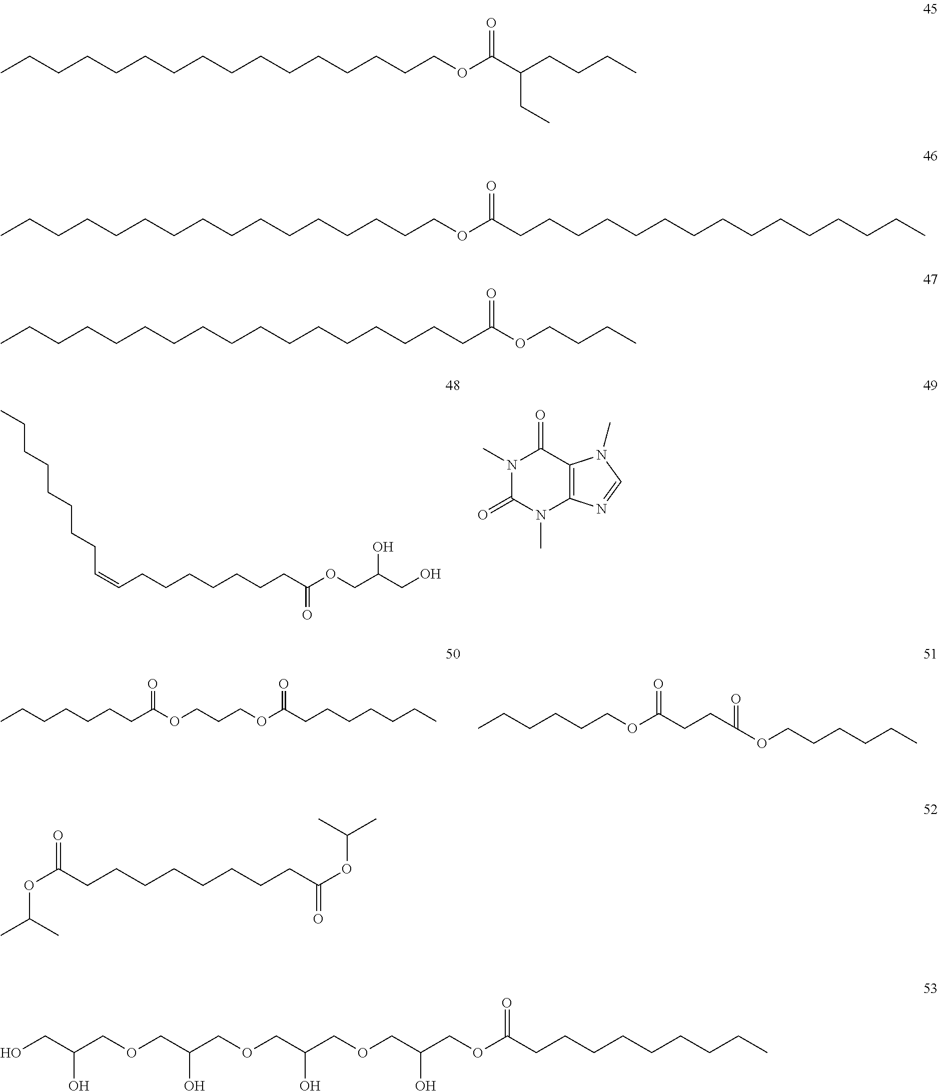

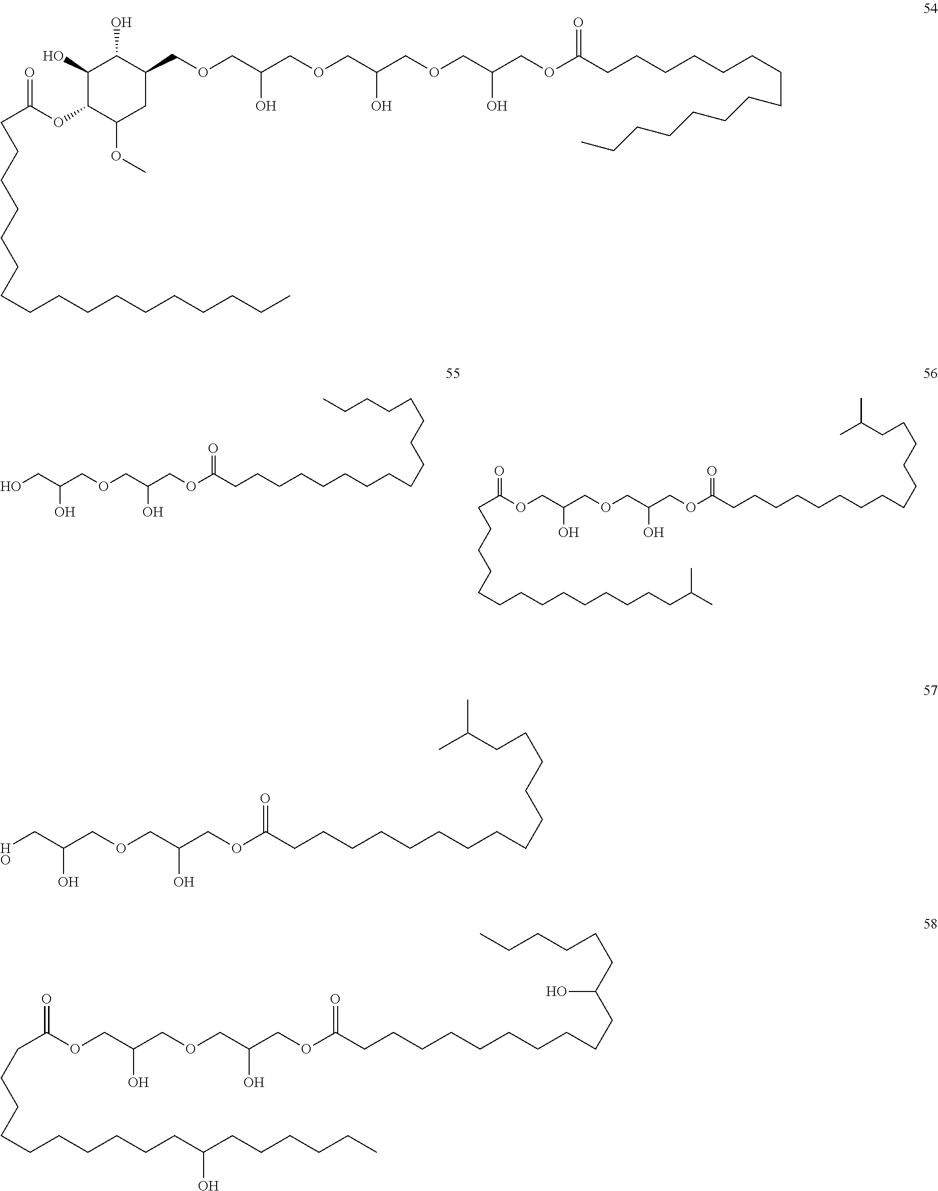 [SMILES: CCCCCCCCCCCCCCCCCC(=O)O[C@H]1C(OC)C[C@H](COCC(O)COCC(O)COCC(O)COC(=O)CCCCCCCCCCCCCCC)[C@@H](O)[C@@H]1O ; CCCCCCCCCCCCCCCCCC(=O)OCC(O)COCC(O)CO ; CC(C)CCCCCCCCCCCCCCC(=O)OCC(O)COCC(O)COC(=O)CCCCCCCCCCCCCCC(C)C ; CC(C)CCCCCCCCCCCCCCC(=O)OCC(O)COCC(O)CO ; CCCCCCC(O)CCCCCCCCCCC(=O)OCC(O)COCC(O)COC(=O)CCCCCCCCCCC(O)CCCCCC]

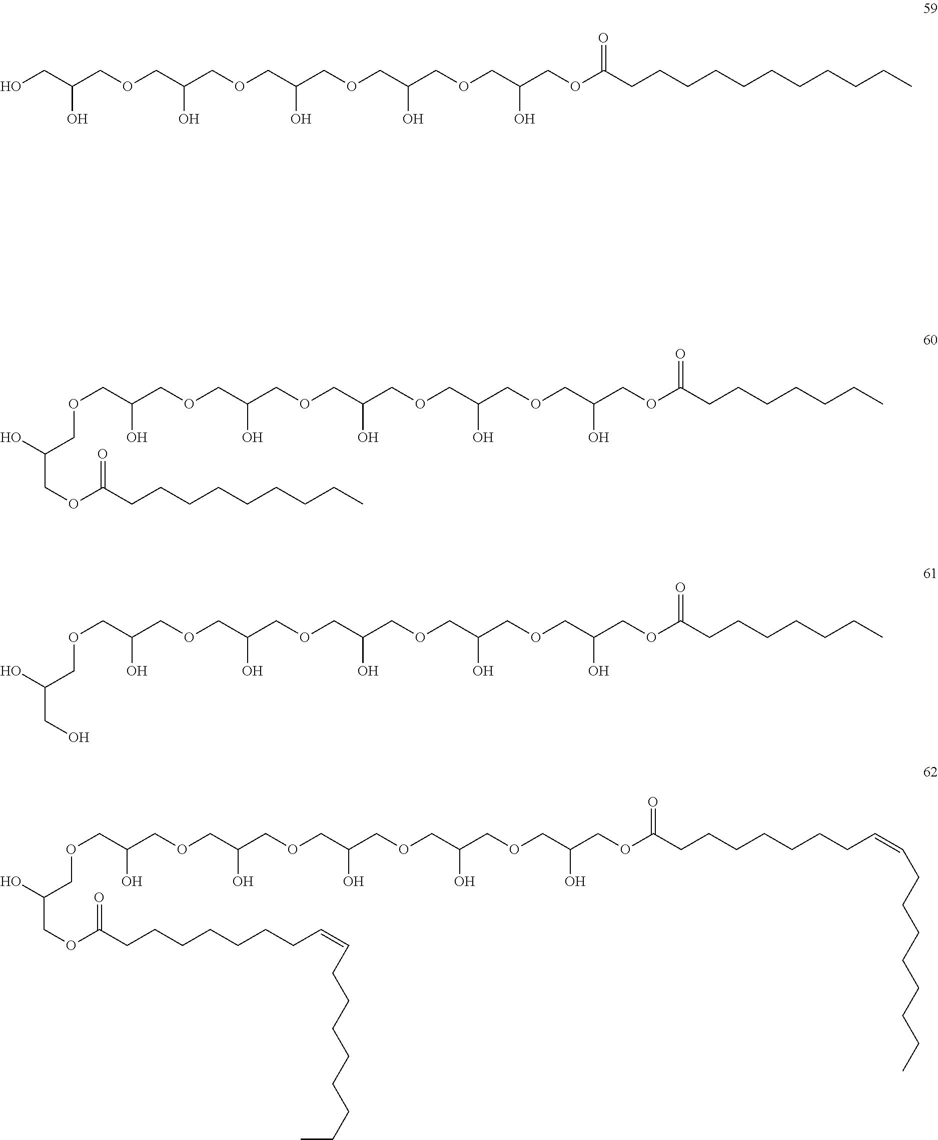 [SMILES: CCCCCCCCCCCC(=O)OCC(O)COCC(O)COCC(O)COCC(O)COCC(O)CO ; CCCCCCCCCC(=O)OCC(O)COCC(O)COCC(O)COCC(O)COCC(O)COCC(O)COC(=O)CCCCCCC ; CCCCCCCC(=O)OCC(O)COCC(O)COCC(O)COCC(O)COCC(O)COCC(O)CO ; CCCCCCCC/C=C\CCCCCCCC(=O)OCC(O)COCC(O)COCC(O)COCC(O)COCC(O)COCC(O)COC(=O)CCCCCCC/C=C\CCCCCCCC]

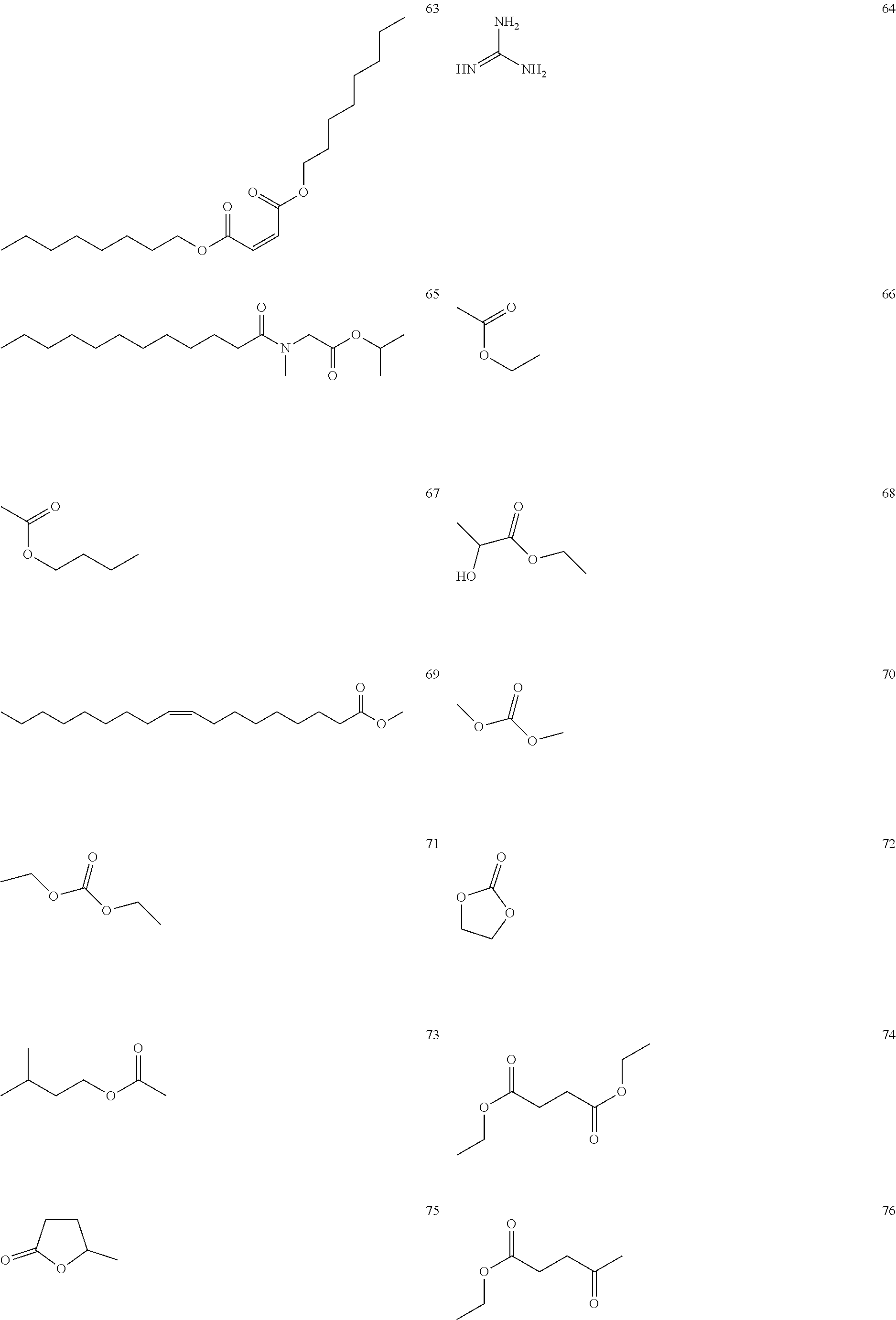

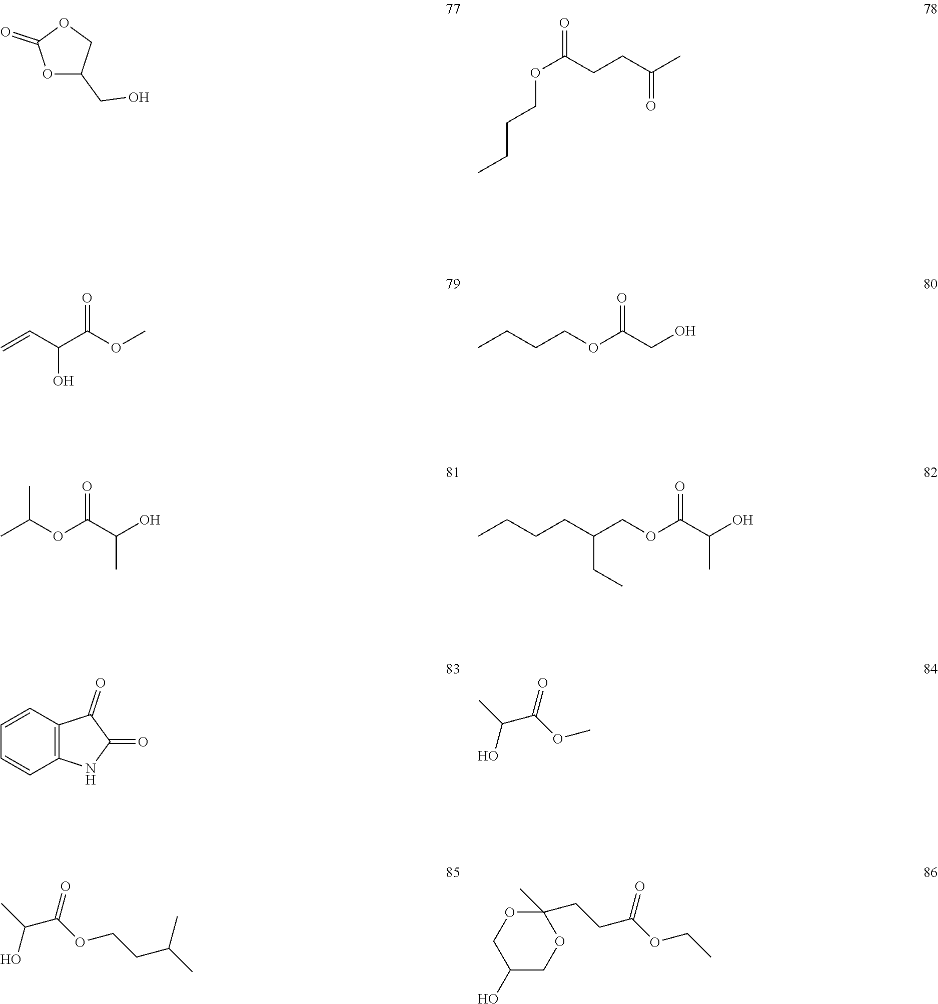

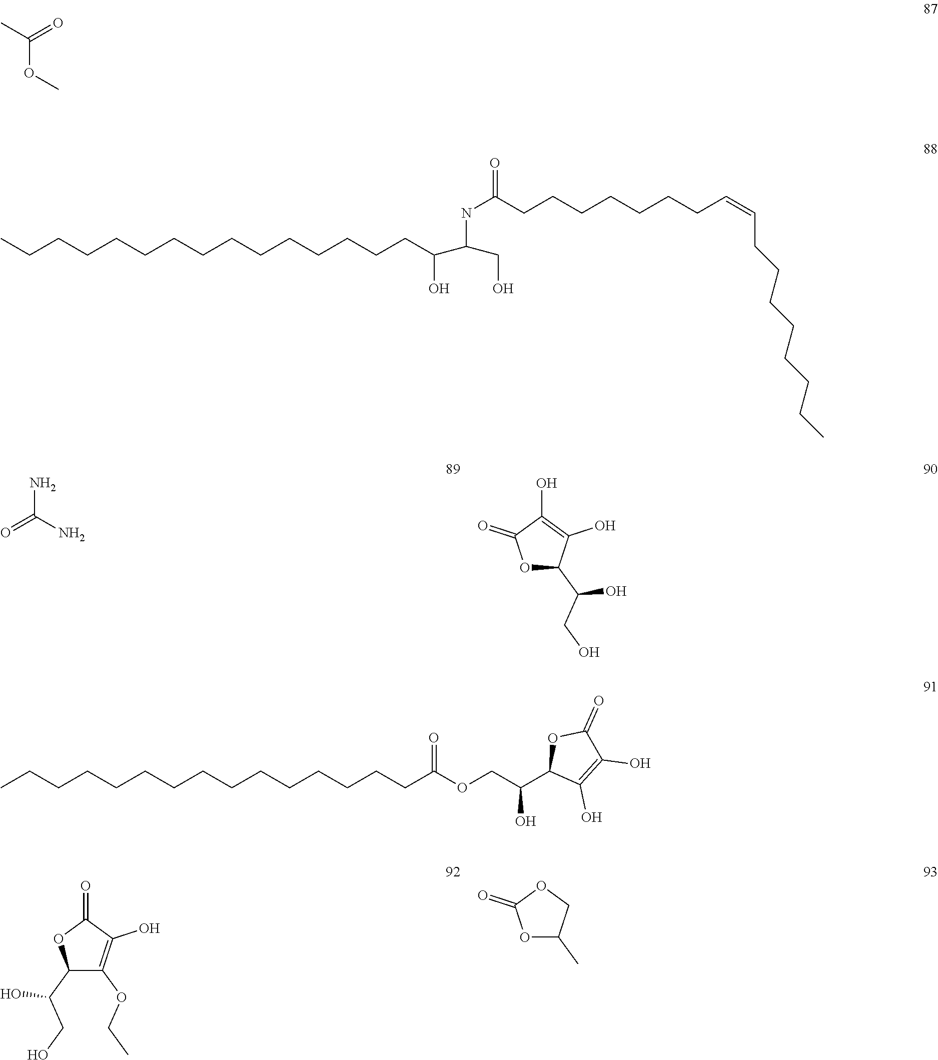

Even more preferentially, the compound(s) iv) are chosen from compounds 1 to 63, salts thereof, optical isomers thereof, geometric isomers thereof, tautomers thereof, solvates thereof, and mixtures thereof.

And even better still, the compound(s) iv) are chosen from compounds 1, 4, 5, 6, 12, 15, 16, 22, 24, 26, 27, 31, 33, 37, 41, 53, 54, 55, 58, 60, 62 and 63, salts thereof, optical isomers thereof, geometric isomers thereof, tautomers thereof, solvates thereof, and mixtures thereof.

The compound(s) iv) are present in the composition preferably in a content ranging from 0.01% to 50%, more preferentially ranging from 1% to 30%, even more preferentially ranging from 2% to 15% by weight, relative to the total weight of the composition.

The composition preferably comprises a total content of magnesium carbonate of less than 5% by weight, more preferentially of less than 1% by weight, even more preferentially of less than 0.1% by weight, most preferentially of less than 0.01% by weight, and better still of less than 0.001% by weight.

According to a particularly preferred embodiment, the composition is devoid of magnesium carbonate.

The composition comprises a total content of persulfates of less than 5% by weight, preferably of less than 1% by weight, more preferentially of less than 0.1% by weight, even more preferentially of less than 0.01% by weight, most preferentially of less than 0.001% by weight.

According to a particularly preferred embodiment, the composition is devoid of persulfates.

According to a particular embodiment, the composition also comprises v) one or more colouring agents chosen from direct dyes, oxidation dyes, and mixtures thereof.

When they are present, the colouring agent(s) are preferably present in a total content ranging from 0.001% to 10% by weight, preferably from 0.01% to 4% by weight, more preferentially from 0.1% to 1% by weight, relative to the total weight of the composition.

### Oxidation Dyes

The oxidation dyes are generally chosen from one or more oxidation bases, optionally combined with one or more coupling agents (also known as couplers).

### Oxidation Bases

The composition may optionally comprise one or more oxidation bases advantageously chosen from those conventionally used in the dyeing of keratin fibres.

By way of example, the oxidation bases are chosen from para-phenylenediamines, bis(phenyl)alkylenediamines, para-aminophenols, ortho-aminophenols and heterocyclic bases, and the corresponding addition salts.

The para-phenylenediamines which may be mentioned include, for example, para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-p-hydroxyethyl-para-phenylenediamine, 2-methoxymethyl-para-phenylenediamine, 2-fluoro-para-phenylenediamine, 2-isopropyl-para-phenylenediamine, N—(β-hydroxypropyl)-para-phenylenediamine, 2-hydroxymethyl-para-phenylenediamine, N,N-dimethyl-3-methyl-para-phenylenediamine, N-ethyl-N—(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-p-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine and the corresponding addition salts with an acid.

Preference is in particular given, among the abovementioned para-phenylenediamines, to para-phenylenediamine, para-toluenediamine, 2-isopropyl-para-phenylenediamine, 2-p-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine and the corresponding addition salts with an acid.

The bis(phenyl)alkylenediamines which may be mentioned include, for example, N,N′-bis((3-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane and the corresponding addition salts.

The para-aminophenols which are mentioned include, for example, para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol and the corresponding addition salts with an acid.

The ortho-aminophenols which may be mentioned include, for example, 2-aminophenol, 2-amino-5-methylphenol, 2-amino-6-methylphenol and 5-acetamido-2-aminophenol and the corresponding addition salts.

The heterocyclic bases which may be mentioned include, for example, pyridine, pyrimidine and pyrazole derivatives.

The pyridine derivatives which may be mentioned include the compounds for example described in patents GB 1 026 978 and GB 1 153 196, for example 2,5-diaminopyridine, 2-(4-methoxyphenyl)amino-3-aminopyridine and 3,4-diaminopyridine and the corresponding addition salts.

Other pyridine oxidation bases which are of use in the present invention are the 3-aminopyrazolo[1,5-a]pyridine oxidation bases or the corresponding addition salts described, for example, in patent application FR 2 801 308. Examples which may be mentioned include pyrazolo[1,5-a]pyrid-3-ylamine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-β-hydroxyethoxy-3-aminopyrazolo[1,5-a]pyridine and 2-(4-dimethylpiperazinium-1-yl)-3-aminopyrazolo[1,5-a]pyridine, and the corresponding addition salts.

More particularly, the oxidation bases which are of use in the present invention are chosen from 3-aminopyrazolo[1,5-a]pyridines which are preferably substituted on carbon atom 2 by:
a) a (di)(C₁-C₆)(alkyl)amino group, it being possible for said alkyl group to be substituted by at least one hydroxyl, amino or imidazolium group;
b) an optionally cationic 5- to 7-membered heterocycloalkyl group containing from 1 to 3 heteroatoms, optionally substituted by one or more (C₁-C₆)alkyl groups, such as a di(C₁-C₄)alkylpiperazinium group; or
c) a (C₁-C₆)alkoxy group optionally substituted by one or more hydroxyl groups, such as a β-hydroxyalkoxy group, and the corresponding addition salts.

The pyrimidine derivatives which may be mentioned include the compounds described, for example, in patents DE 2359399, JP 88-169571, JP 05-63124 and EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their addition salts and their tautomeric forms, when a tautomeric equilibrium exists.

The pyrazole derivatives which may be mentioned include the compounds described in patents DE 3843892 and DE 4133957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole and the corresponding addition salts. Use may also be made of 4,5-diamino-1-(β-methoxyethyl)pyrazole.

A 4,5-diaminopyrazole will preferably be used and more preferentially still 4,5-diamino-1-((3-hydroxyethyl)pyrazole and/or a corresponding salt.

The pyrazole derivatives which may also be mentioned comprise diamino-N,N-dihydropyrazolopyrazolones and in particular those described in patent application FR-A-2 886 136, such as the following compounds and the corresponding addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-di(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one and 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol-1-one and/or a corresponding salt.

Use will preferably be made, as heterocyclic bases, of 4,5-diamino-1-(β-hydroxyethyl)pyrazole and/or 2,3-diamino-6,7-dihydro-1H,5H-pyrazolo[1,2-a]pyrazol-1-one and/or a corresponding salt.

### Coupling Agents

The composition may optionally comprise one or more coupling agents advantageously chosen from those conventionally used in the dyeing of keratin fibres.

Mention may in particular be made, among these coupling agents, of meta-phenylenediamines, meta-aminophenols, meta-diphenols, naphthalene-based coupling agents and heterocyclic coupling agents, and also the corresponding addition salts.

Mention may be made, for example, of 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-p-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N—(β-hydroxyethyl)amino-3,4-methylenedioxybenzene, 2,6-bis(β-hydroxyethylamino)toluene, 6-hydroxyindoline, 2,6-dihydroxy-4-methylpyridine, 1H-3-methylpyrazol-5-one, 1-phenyl-3-methylpyrazol-5-one, 2,6-dimethylpyrazolo[1,5-b][1,2,4]triazole, 2,6-dimethyl[3,2-c][1,2,4]triazole, 6-methylpyrazolo[1,5-a]benzimidazole, 2-methyl-5-aminophenol, 5-N—(β-hydroxyethyl)amino-2-methylphenol, 3-aminophenol and 3-amino-2-chloro-6-methylphenol, the corresponding addition salts with an acid and the corresponding mixtures.

In general, the addition salts of oxidation bases and of coupling agents which can be used in the context of the invention are chosen in particular from the addition salts with an acid, such as the hydrochlorides, hydrobromides, sulfates, citrates, succinates, tartrates, lactates, tosylates, benzenesulfonates, phosphates and acetates.

The oxidation base(s) each advantageously represent(s) from 0.001% to 10% by weight relative to the total weight of the composition, and preferably from 0.005% to 5% by weight relative to the total weight of the composition.

The coupling agent(s), if they are present, each advantageously represent(s) from 0.001% to 10% by weight relative to the total weight of the composition, and preferably from 0.005% to 5% by weight relative to the total weight of the composition.

### Direct Dyes

The composition may also comprise one or more direct dyes.

The direct dyes may be neutral, cationic or anionic direct dyes, preferably neutral or cationic direct dyes.

The direct dyes may be neutral, cationic or anionic direct dyes chosen from: acridines; acridones; anthranthrones; anthrapyrimidines; anthraquinones; azines; (poly)azos or azos, hydrazono or hydrazones, in particular arylhydrazones; azomethines; benzanthrones; benzimidazoles; benzimidazolones; benzindoles; benzoxazoles; benzopyrans; benzothiazoles; benzoquinones; bis-isoindolines; carboxanilides; coumarins; cyanines, such as (di)azacarbocyanines, (di)azahemicyanines, hemicyanines, or tetraazacarbocyanines; (di)azines; bis-azines; (di)oxazines; (di)thiazines; (di)phenylamines; (di)phenylmethanes; (di)ketopyrrolopyrroles; flavonoids, such as flavanthrones and flavones; fluorindines; formazans; indamines; indanthrones; indigoids, thioindigoids and pseudoindigoids; indophenols; indoanilines; isoindolines; isoindolinones; isoviolanthrones; lactones; (poly)methines, such as dimethines of stilbene or styryl types; naphthalimides; naphthanilides; naphtholactams; naphthoquinones; nitro, notably nitro(hetero)aromatics; oxadiazoles; oxazines; perilones; perinones; perylenes; phenazines; phenoxazine; phenothiazines; phthalocyanine; polyenes/carotenoids; porphyrins; pyranthrones; pyrazolanthrones; pyrazolones; pyrimidinoanthrones; pyronines; quinacridones; quinolines; quinophthalones; squaranes; tetrazolines; thiazines; thiopyronines; triarylmethanes or xanthenes and natural direct dyes. Preferably, the direct dyes are chosen from anthraquinones, (poly)azos, azomethines and stilbenes, more preferentially from anthraquinones.

The direct dyes may be chosen in particular from neutral, cationic or anionic nitrobenzene direct dyes, neutral, cationic or anionic azo direct dyes, neutral, cationic or anionic tetraazapentamethine dyes, cationic or anionic quinone dyes and in particular neutral, cationic or anionic anthraquinone dyes, neutral, cationic or anionic azine direct dyes, neutral, cationic or anionic triarylmethane direct dyes, neutral, cationic or anionic azomethine direct dyes and natural direct dyes. Preferably, the direct dyes are chosen from neutral or anionic anthraquinone dyes and stilbenes.

As neutral, anionic or cationic direct dyes that may be used in the present invention, mention may be made of the following dyes: acridines; acridones; anthranthrones; anthrapyrimidines; anthraquinones; azines; (poly)azos, hydrazono or hydrazones, in particular arylhydrazones; azomethines; benzanthrones; benzimidazoles; benzimidazolones; benzindoles; benzoxazoles; benzopyrans; benzothiazoles; benzoquinones; bisazines; bis-isoindolines; carboxanilides; coumarins; cyanines, such as azacarbocyanines, diazacarbocyanines, diazahemicyanines, hemicyanines, or tetraazacarbocyanines; diazines; diketopyrrolopyrroles; dioxazines; diphenylamines; diphenylmethanes; dithiazines; flavonoids, such as flavanthrones and flavones; fluorindines; formazans; indamines; indanthrones; indigoids and pseudoindigoids; indophenols; indoanilines; isoindolines; isoindolinones; isoviolanthrones; lactones; (poly)methines, such as dimethines of stilbene or styryl types; naphthalimides; naphthanilides; naphtholactams; naphthoquinones; nitro, notably nitro(hetero)aromatics; oxadiazoles; oxazines; perilones; perinones; perylenes; phenazines; phenoxazine; phenothiazines; phthalocyanine; polyenes/carotenoids; porphyrins; pyranthrones; pyrazolanthrones; pyrazolones; pyrimidinoanthrones; pyronines; quinacridones; quinolines; quinophthalones; squaranes; tetrazoles; thiazines; thioindigo; thiopyronines; triarylmethanes or xanthenes.

### Neutral Direct Dyes

The direct dyes may be neutral direct dyes, preferably chosen from the hydrazono dyes of formulae (IIIa) and (III′a), the azo and styryl dyes (IVa), the diazo and distyryl dyes (IV′a) and (IV″a), the anthraquinone dyes (Va) and the azomethine dyes (VIa) and (VI′a) below, and mixtures thereof:

| | | | |
|---|---|---|---|
| Ar″—C(R^{a})═N—N(R^{b})—Ar | Ar″—N(Rª)—N═C(R^{b})—Ar | Ar—T═T′—Ar″ | Ar″—T═T′—Ar′—T′═T—Ar |
| (IIIa) | (III′a) | (IVa) | (IV′a) |

| | |
|---|---|
| Ar—T═T—L—T′═T—Ar | 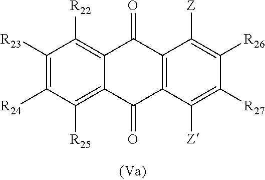 |

| | |
|---|---|
| 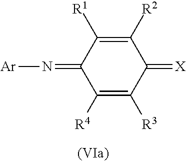 | 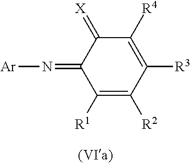 |

in which formulae (IIIa), (III′a), (IVa), (IV′a), (IV″a), (Va), (VIa) and (VI′a):
Ar represents an aryl group, such as phenyl or naphthyl, substituted by at least one electron-donating group such as i) optionally substituted (C₁-C₈)alkyl, ii) optionally substituted (C₁-C₈)alkoxy, iii) (di)(C₁-C₈)(alkyl)amino optionally substituted on the alkyl group(s) by a hydroxyl group, iv) aryl(C₁-C₈)alkylamino, v) optionally substituted N—(C₁-C₈)alkyl-N-aryl(C₁-C₈)alkylamino, or Ar represents a julolidine group;
Ar′ represents an optionally substituted divalent (hetero)arylene group, such as phenylene, particularly para-phenylene, or naphthalene, which is optionally substituted, preferably by one or more (C₁-C₈)alkyl, hydroxyl or (C₁-C₈)alkoxy groups;
Ar″ represents a (hetero)aryl group, which is optionally substituted, preferably by at least i) an electron-withdrawing group such as nitro, nitroso, —C(X)—X′—R′ or ii) a (di)(C₁-C₆)(alkyl)amino group, iii) hydroxyl, iv) (C₁-C₆)alkoxy; (hetero)aryl is particularly chosen from imidazolyl, triazolyl, indolyl or pyridyl or phenyl optionally substituted by at least one group chosen from nitro, nitroso and amino, preferably substituted in the para position of the phenyl group;
X, X′ and X″, which may be identical or different, represent an oxygen or sulfur atom, or a group NR″, preferably an oxygen atom;
R¹, R², R³ and R⁴, which may be identical or different, represent a hydrogen or halogen atom, or a group chosen from hydroxyl, thiol, (C₁-C₄)alkyl, (C₁-C₄)alkoxy, (di)(C₁-C₄)(alkyl)amino, nitro and nitroso;
R′ and R″ represent a (C₁-C₄)alkyl group;
R^{a} and R^{b}, which may be identical or different, represent a hydrogen atom or a (C₁-C₈)alkyl group, which is optionally substituted, preferably by a hydroxyl group; or, as a variant, the substituent R^{a} with a substituent of Ar″ and/or R^{b} with a substituent of Ar and/or R^{a} with R^{b} form, together with the atoms that bear them, a (hetero)cycloalkyl; in particular, R^{a} and R^{b} represent a hydrogen atom or a (C₁-C₄)alkyl group, which is optionally substituted by a hydroxyl group;
T and T′, which may be identical or different, represent a group C(R^{a}) or N, preferably N; and
L represents a divalent group -ALK-, —C(X)-ALK-, -ALK—C(X)— or —C(X)-ALK—C(X′)—, with ALK representing a linear or branched (C₁-C₆)alkylene group, such as methylene, and X and X′ as defined above;
R₂₂, R₂₃, R₂₄, R₂₅, R₂₆ and R₂₇, which may be identical or different, represent a hydrogen or halogen atom, or a group chosen from:
(C₁-C₆)alkyl;
hydroxyl, mercapto;
(C₁-C₆)alkoxy, (C₁-C₆)alkylthio;
aryloxy or arylthio;
aryl(C₁-C₆)(alkyl)amino;
(di)(C₁-C₆)(alkyl)amino;
(di)(hydroxy(C₁-C₆)alkyl)amino;
Z′ represents a hydrogen atom or a group NR₂₈R₂₉ with R₂₈ and R₂₉, which may be identical or different, representing a hydrogen atom or a group chosen from:
(C₁-C₆)alkyl;
polyhydroxy(C₁-C₆)alkyl such as hydroxyethyl;
aryl optionally substituted by one or more groups, particularly i) (C₁-C₆)alkyl; iii) R^{o}—C(X)—X′—, R^{o}—X′—C(X)— or R^{o}—X′—C(X)—X″— with R^{o} representing a (C₁-C₆)alkyl group and X, X′ and X″ as defined above; iv) a sulfonate;
cycloalkyl; notably cyclohexyl;
Z represents a group chosen from hydroxyl and NR′₂₈R′₂₉ with R′₂₈ and R′₂₉, which may be identical or different, representing the same atoms or groups as R₂₈ and R₂₉ as defined above.

The direct dyes of formula (IV″a) are preferably of formula (IV′″a)

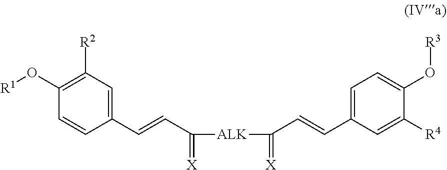

in which formula (IV′″a):
R¹ and R³, which may be identical or different, preferably identical, represent a hydrogen atom, a (C₁-C₄)alkyl group such as methyl or a sugar such as glucosyl, preferably a hydrogen atom;
R² and R⁴, which may be identical or different, preferably identical, represent a hydrogen atom, a (C₁-C₄)alkyl or (C₁-C₄)alkoxy group or an —O-sugar group such as —O— glucosyl, preferably (C₁-C₄)alkoxy; such as methoxy;
X, which may be identical or different, preferably identical, represent an oxygen or sulfur atom or N—R with R representing a hydrogen atom or a group, preferably an oxygen atom;
ALK represents a (C₁-C₄)alkylene group such as methylene or ethylene, preferably methylene.

The direct dyes of formula (IV″a) may be derived from curcumin, demethoxycurcumin and bis-demethoxycurcumin.

Preferably, the direct dyes are chosen from the direct dyes of formulae (IV″a) and (IV′″a) and mixtures thereof as defined above.

According to a particularly preferred embodiment, the direct dyes are neutral direct dyes chosen from the following compounds (A) to (G) and mixtures thereof:

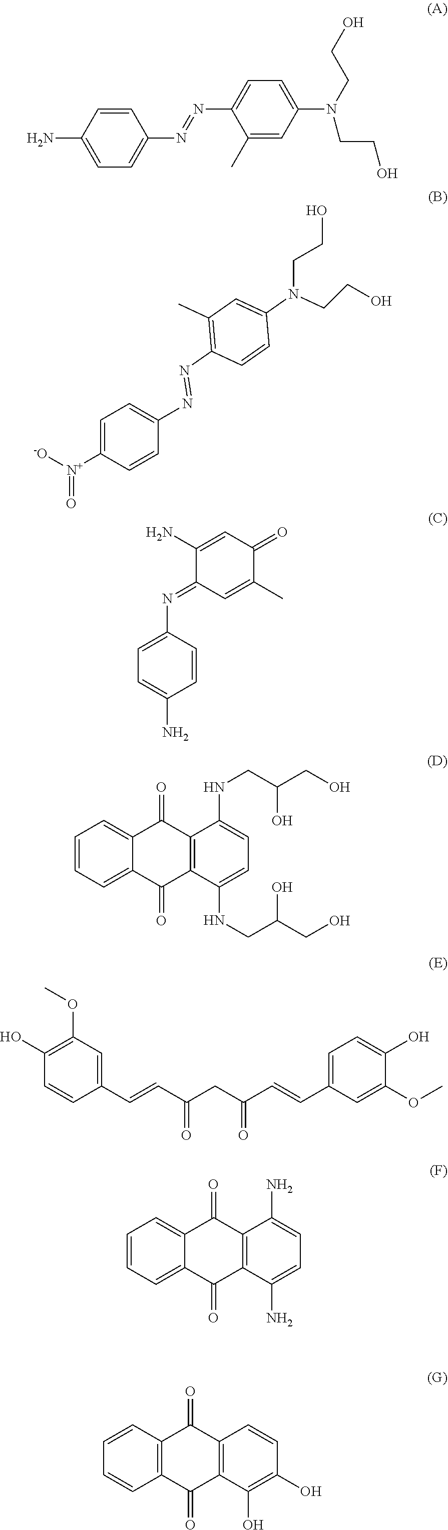

preferably from the compounds (E), (F) and (G) and mixtures thereof, more preferentially from the compounds (E) and (G) and mixtures thereof.

### Cationic Direct Dyes

The direct dyes may be chosen from direct dyes which are cationic or commonly referred to as “basic dyes” for their affinity with acidic substances notably including in their structure at least one endocyclic or exocyclic cationic or cationizable group.

As cationic azo dyes that can be used in the present invention, mention may be made particularly of the cationic dyes described in Kirk-Othmer's Encyclopaedia of Chemical Technology, “Dyes, Azo”, J. Wiley & Sons, updated on 19 Apr. 2010.

Mention may also be made of the cationic azo dyes described in patent applications WO 95/15144, WO 95/01772 and EP 714954.

Mention may also be made of the cationic azo dyes described in the Colour Index International 3rd Edition, notably of the following compounds: Basic Red 22; Basic Red 76; Basic Yellow 57; Basic Brown 16; Basic Brown 17.

Among the cationic quinone dyes, those mentioned in the Colour Index International 3rd Edition are suitable for use and, among these, mention may be made, inter alia, of the following dyes: Basic Blue 22; Basic Blue 99.

Among the azine dyes that are suitable for use, mention may be made of those listed in the Colour Index International 3rd Edition, and for example the following dyes: Basic Blue 17, Basic Red 2.

Among the cationic triarylmethane dyes that may be used according to the invention, mention may be made, in addition to those listed in the Colour Index International 3rd Edition, of the following dyes: Basic Green 1, Basic Violet 3, Basic Violet 14, Basic Blue 7, Basic Blue 26.

Mention may also be made of the direct dyes in documents U.S. Pat. No. 5,888,252, EP 1133975, WO 03/029359, EP 860636, WO 95/01772, WO 95/15144 and EP 714954.

Mention may also be made of those listed in the encyclopaedia “The Chemistry of Synthetic Dyes” by K. Venkataraman, 1952, Academic Press, volumes 1 to 7, in the “Kirk-Othmer Encyclopaedia of Chemical Technology”, in the chapter “Dyes and Dye Intermediates”, 1993, Wiley and Sons, and in various chapters of “Ullmann's Encyclopaedia of Industrial Chemistry”, 7th edition, Wiley and Sons.

Preferably, the cationic direct dyes are chosen from those resulting from dyes of azo and hydrazono type.

The cationic direct dyes may be cationic azo dyes, as described in EP 850636, FR 2788433, EP 920856, WO 9948465, FR 2757385, EP 850637, EP 918053, WO 9744004, FR 2570946, FR 2285851, DE 2538363, FR 2189006, FR 1560664, FR 1540423, FR 1567219, FR 1516943, FR 1221122, DE 4220388, DE 4137005, WO 0166646, U.S. Pat. No. 5,708,151, WO 9501772, WO 515144, GB 1195386, U.S. Pat. Nos. 3,524,842, 5,879,413, EP 1062940, EP 1133976, GB 738585, DE 2527638, FR 2275462, GB 1974-27645, Acta Histochem. (1978), 61(1), 48-52; Tsitologiya (1968), 10(3), 403-5; Zh. Obshch. Khim. (1970), 40(1), 195-202; Ann. Chim. (Rome) (1975), 65(5-6), 305-14; Journal of the Chinese Chemical Society (Taipei) (1998), 45(1), 209-211; Rev. Roum. Chim. (1988), 33(4), 377-83; Text. Res. J. (1984), 54(2), 105-7; Chim. Ind. (Milan) (1974), 56(9), 600-3; Khim. Tekhnol. (1979), 22(5), 548-53; Ger. Monatsh. Chem. (1975), 106(3), 643-8; MRL Bull. Res. Dev. (1992), 6(2), 21-7; Lihua Jianyan, Huaxue Fence (1993), 29(4), 233-4; Dyes Pigm. (1992), 19(1), 69-79; Dyes Pigm. (1989), 11(3), 163-72.

Preferably, the cationic direct dyes comprise a quaternary ammonium group; more preferentially, the cationic charge is endocyclic. These cationic groups are, for example, a cationic group:
bearing a (di/tri)(C₁-C₈)alkylammonium exocyclic charge, or
bearing an endocyclic charge, such as comprising a cationic heteroaryl group chosen from: acridinium, benzimidazolium, benzobistriazolium, benzopyrazolium, benzopyridazinium, benzoquinolium, benzothiazolium, benzotriazolium, benzoxazolium, bipyridinium, bis-tetrazolium, dihydrothiazolium, imidazopyridinium, imidazolium, indolium, isoquinolium, naphthoimidazolium, naphthoxazolium, naphthopyrazolium, oxadiazolium, oxazolium, oxazolopyridinium, oxonium, phenazinium, phenooxazolium, pyrazinium, pyrazolium, pyrazoyltriazolium, pyridinium, pyridinoimidazolium, pyrrolium, pyrylium, quinolium, tetrazolium, thiadiazolium, thiazolium, thiazolopyridinium, thiazoylimidazolium, thiopyrylium, triazolium or xanthylium.

Mention may be made of the cationic hydrazono direct dyes of formulae (IIb) and (IIIb) and the azo direct dyes of formulae (IVb) and (Vb) below:

Het⁺-C(R^{a})=N—N(R_{b})—Ar,Q⁻ (IIb);

Het⁺-N(R^{a})—N═C(R_{b})—Ar,Q⁻ (IIIb);

Het⁺—N═N—Ar,Q (IVb);

Ar⁺—N═N—Ar″,Q⁻ (Vb);

in which formulae (IIb) to (Vb):
Het⁺ represents a cationic heteroaryl group, preferentially bearing an endocyclic cationic charge, such as imidazolium, indolium or pyridinium, which is optionally substituted, preferentially by at least one (C₁-C₈)alkyl group such as methyl;
Ar⁺ represents an aryl group, such as phenyl or naphthyl, bearing an exocyclic cationic charge, preferentially ammonium, particularly tri(C₁-C₈)alkylammonium, such as trimethylammonium;
Ar represents an aryl group, notably phenyl, which is optionally substituted, preferentially by one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) by a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially by one or more (C₁-C₈)alkyl, hydroxyl, (di)(C₁-C₈)(alkyl)amino, (C₁-C₈)alkoxy or phenyl groups;
Rₐ and R_{b}, which may be identical or different, represent a hydrogen atom or a (C₁-C₈)alkyl group which is optionally substituted, preferentially by a hydroxyl group;
or else the substituent Rₐ with a substituent of Het⁺ and/or R_{b} with a substituent of Ar and/or Rₐ with R_{b} form, together with the atoms which bear them, a (hetero)cycloalkyl; in particular, Rₐ and R_{b} represent a hydrogen atom or a (C₁-C₄)alkyl group optionally substituted by a hydroxyl group;
Q⁻ represents an anionic counterion such as a halide, an alkyl sulfate or an alkylsulfonate.

In particular, mention may be made of the azo and hydrazono direct dyes bearing an endocyclic cationic charge of formulae (lib) to (Vb) as defined above. More particularly, mention may be made of the cationic direct dyes of formulae (lib) to (Vb) bearing an endocyclic cationic charge described in patent applications WO 95/15144, WO 95/01772 and EP 714954.

Preferably, mention may be made of the following direct dyes:

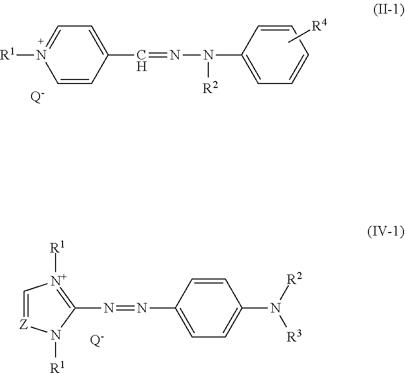

in which formulae (II-1) and (IV-1):
R¹ represents a (C₁-C₄)alkyl group, such as methyl;
R² and R³, which may be identical or different, represent a hydrogen atom or a (C₁-C₄)alkyl group, such as methyl;
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) by a hydroxyl group; in particular, R⁴ is a hydrogen atom;
Z represents a CH group or a nitrogen atom, preferentially CH;
Q⁻ is an anionic counterion as defined above, in particular a halide, such as chloride, or an alkyl sulfate, such as methyl sulfate or mesyl.

Particularly, the dyes of formulae (II-1) and (IV-1) are chosen from Basic Red 51, Basic Yellow 87 and Basic Orange 31 or derivatives thereof:

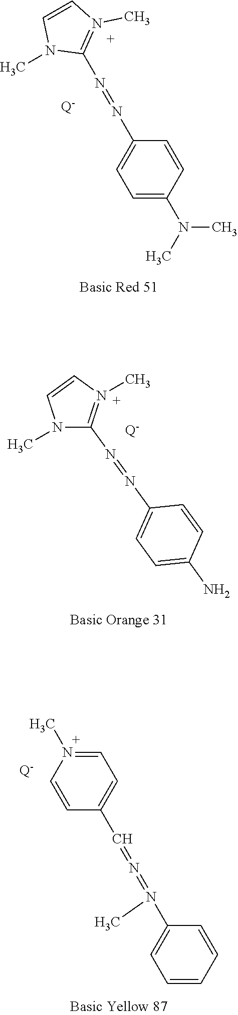

with Q′ being an anionic counterion as defined above, in particular a halide, such as chloride, or an alkyl sulfate, such as methyl sulfate or mesyl.

### Fluorescent Dyes

The direct dyes may be chosen from fluorescent direct dyes.

As examples of fluorescent dyes that may be used in the present invention, mention may be made of neutral, anionic or cationic dyes chosen from the following dyes: acridines, acridones, benzanthrones, benzimidazoles, benzimidazolones, benzindoles, benzoxazoles, benzopyrans, benzothiazoles, coumarins, difluoro{2-[(2H-pyrrol-2-ylidene-kN)methyl]-1H-pyrrolato-kN}borons (BODIPY®), diketopyrrolopyrroles, fluorindines, (poly)methines (notably cyanines and styryls/hemicyanines), naphthalimides, naphthanilides, naphthylamines (such as dansyls), oxadiazoles, oxazines, perilones, perinones, perylenes, polyenes/carotenoids, squaranes, stilbenes, xanthenes.

Mention may also be made of the fluorescent dyes described in documents EP 1133975, WO 03/029359, EP 860636, WO 95/01772, WO 95/15144 and EP 714954 and those listed in the encyclopaedia “The Chemistry of Synthetic Dyes” by K. Venkataraman, 1952, Academic Press, volumes 1 to 7, in the “Kirk-Othmer Encyclopaedia of Chemical Technology”, in the chapter “Dyes and Dye Intermediates”, 1993, Wiley and Sons, and in various chapters of “Ullmann's Encyclopaedia of Industrial Chemistry”, 7th edition, Wiley and Sons, and in the handbook—“A Guide to Fluorescent Probes and Labeling Technologies”, 10th Ed., Molecular Probes/Invitrogen—Oregon 2005, circulated on the Internet or in the preceding printed editions.

According to a preferred variant, the fluorescent dye(s) are cationic polymethines and comprise at least one quaternary ammonium group, such as those of the following formula (Vb): W⁺—[C(R_{c})═C(R_{d})]_{m′}—Ar, Q⁻

in which formula (Vb):
W⁺ represents a cationic heterocyclic or heteroaryl group, particularly comprising a quaternary ammonium optionally substituted by one or more (C₁-C₈)alkyl groups optionally substituted in particular by one or more hydroxyl groups;
Ar represents an aryl group, such as phenyl or naphthyl, which are optionally substituted, preferentially by i) one or more halogen atoms, such as chlorine or fluorine; ii) one or more (C₁-C₈)alkyl, preferably (C₁-C₄)alkyl, groups, such as methyl; iii) one or more hydroxyl groups; iv) one or more (C₁-C₈)alkoxy groups, such as methoxy; v) one or more hydroxy(C₁-C₈)alkyl groups, such as hydroxyethyl, vi) one or more amino or (di)(C₁-C₈)alkylamino groups, preferably with the alkyl part C₁-C₄, optionally substituted by one or more hydroxyl groups, such as (di)hydroxyethylamino, vii) one or more acylamino groups; viii) one or more heterocycloalkyl groups, such as piperazinyl, piperidinyl or 5- or 6-membered heteroaryl, such as pyrrolidinyl, pyridinyl and imidazolinyl;
m′ represents an integer ranging from 1 to 4; preferably, m′ is equal to 1 or 2; more preferentially, m′=1;
R_{c} and R_{d}, which may be identical or different, represent a hydrogen atom or an optionally substituted (C₁-C₈)alkyl group, preferably an optionally substituted (C₁-C₄)alkyl group, or alternatively R_{c} contiguous with W⁺ and/or R_{d} contiguous with Ar form, with the atoms which bear them, a (hetero)cycloalkyl; in particular, R_{c} is contiguous with W⁺ and they form a (hetero)cycloalkyl, such as cyclohexyl;
Q⁻ is an anionic counterion as defined above.

### Anionic Dyes

The direct dyes may be chosen from anionic direct dyes or dyes commonly referred to as “acid” direct dyes for their affinity with alkaline substances.

The term “anionic direct dyes” means any direct dye comprising in its structure at least one CO₂R or SO₃R substituent with R denoting a hydrogen atom or a cation originating from a metal or from an amine, or an ammonium ion. The anionic dyes may be chosen from acid nitro direct dyes, acid azo dyes, acid azine dyes, acid triarylmethane dyes, acid indoamine dyes, acid anthraquinone dyes, indigoids and acid natural dyes.

Preferably, the anionic direct dyes are acid anthraquinone dyes.

The direct dyes may be anionic direct dyes preferably chosen from the following dyes of formulae (III) (III′), (IV), (IV′), (V), (V′), (VI), (VI′), (VII), (VIII), (IX) and (X) and their mixtures:
a) the diaryl anionic azo dyes of formula (III) or (III′):

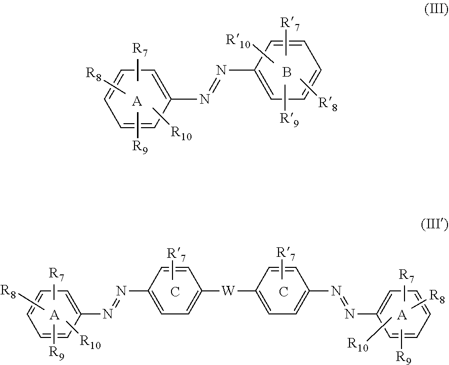

in which formulae (III) and (III′):
   R₇, R₈, R₉, R₁₀, R′₇, R′₈, R′₉ and R′₁₀, which may be identical or different, represent a hydrogen atom or a group chosen from:
   (C₁-C₆)alkyl;
   (C₁-C₆)alkoxy, (C₁-C₆)alkylthio;
   hydroxyl, mercapto;
   nitro, nitroso;
   R^{o}—C(X)—X′—, R^{o}—X′—C(X)—, R^{o}—X′—C(X)—X″— with R^{o} representing a hydrogen atom or a (C₁-C₆)alkyl or aryl group, such as phenyl; X, X′ and X″, which may be identical or different, representing an oxygen or sulfur atom or NR with R representing a hydrogen atom or a (C₁-C₆)alkyl group;
   (O)₂S(O⁻)—, M⁺, with M⁺ representing a hydrogen atom or a cationic counterion;
   (O)CO⁻, M⁺, with M⁺ as defined above;
   R′″—S(O)₂—, with R″ representing a hydrogen atom or an alkyl, aryl, (di)(C₁-C₆)(alkyl)amino or aryl(C₁-C₆)(alkyl)amino group; preferentially a phenylamino or phenyl group;
   R′″—S(O)₂—X′— with R′″ representing a (C₁-C₆)alkyl group or an aryl group which is optionally substituted, and X′ as defined above;
   (di)(C₁-C₆)(alkyl)amino;
   aryl(C₁-C₆)(alkyl)amino, optionally substituted by one or more groups chosen from i) nitro; ii) nitroso; iii) (O)₂S(O⁻)—, M⁺, and iv) (C₁-C₆)alkoxy, with M⁺ as defined above;
   optionally substituted heteroaryl; preferentially a benzothiazolyl group;
   cycloalkyl; in particular cyclohexyl;
   Ar—N═N— with Ar representing an optionally substituted aryl group; preferentially a phenyl optionally substituted by one or more alkyl, (O)₂S(O⁻)—, M⁺, or phenylamino groups;
   or else two contiguous groups, R₇ with R₈ or R₈ with R₉ or R₉ with R₁₀, together form a fused benzo group A′; and R′₇ with R′₈ or R′₈ with R′₉ or R′₉ with R′₁₀ together form a fused benzo group B′; with A′ and B′ optionally substituted by one or more groups chosen from i) nitro; ii) nitroso; iii) (O)₂S(O⁻)—, M⁺; iv) hydroxyl; v) mercapto; vi) (di)(alkyl)amino; vii) R^{o}—C(X)—X′—; viii) R^{o}—X′—C(X)—; ix) R^{o}—X′—C(X)—X″—; x) Ar—N═N— and xi) aryl(C₁-C₆)(alkyl)amino which is optionally substituted; with M⁺, R^{o}, X, X′, X″ and Ar as defined above;
   W represents a sigma σ bond, an oxygen or sulfur atom or a divalent group i) —NR— with R as defined above, or ii) methylene —C(Rₐ)(R_{b})—, with Rₐ and R_{b}, which may be identical or different, representing a hydrogen atom or an aryl group, or else Rₐ and R_{b} form, together with the carbon atom which bears them, a spirocycloalkyl; preferentially, W represents a sulfur atom or Rₐ and R_{b} together form a cyclohexyl;
      it being understood that the formulae (III) and (III′) comprise, on one of the rings A, A′, B, B′ or C:
at least one (O)₂S(O⁻)—, M′⁺ group, with M′⁺ representing a cationic counterion; or
at least one (O)CO⁻, M′⁺ group, with M′⁺ representing a cationic counterion;
   preferably at least one sodium sulfonate group.

Mention may be made, as examples of dyes of formula (III), of: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment Red 57, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3, Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2, Food Yellow 3 or Sunset Yellow;

and mention may be made, as examples of dyes of formula (III′), of: Acid Red 111, Acid Red 134 or Acid Yellow 38.
b) the pyrazolone anionic azo dyes of formula (IV) or (IV′)

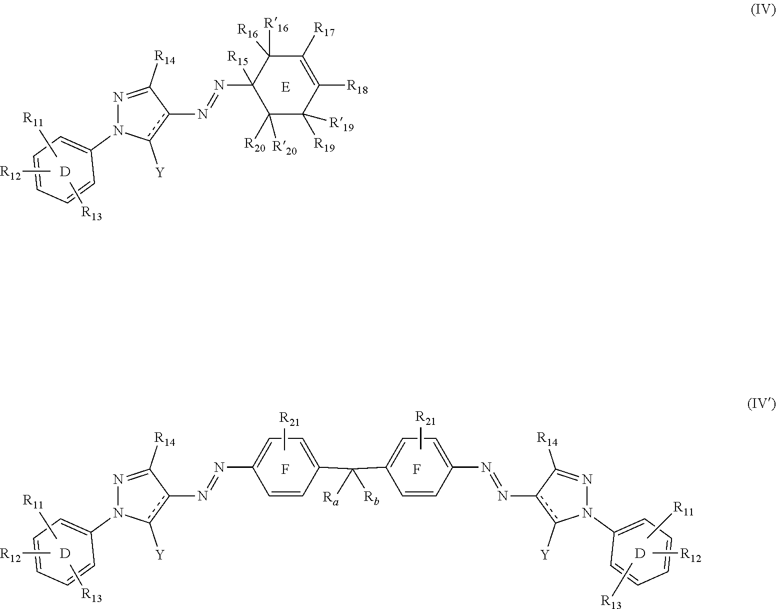

in which formulae (IV) and (IV′):
R₁₁, R₁₂ and R₁₃, which may be identical or different, represent a hydrogen atom, a halogen atom, a (C₁-C₆)alkyl group or an —(O)₂S(O⁻), M⁺ group, with M⁺ as defined above;
R₁₄ represents a hydrogen atom, a (C₁-C₆)alkyl group or an —C(O)O—, M⁺ group, with M⁺ as defined above;
R₁₅ represents a hydrogen atom;
R₁₆ represents an oxo group, in which case R′₁₆ is absent, or else R₁₅ with R₁₆ together form a double bond;
R₁₇ and R₁₈, which may be identical or different, represent a hydrogen atom or a group chosen from:
(O)₂S(O⁻)—, M⁺, with M⁺ as defined above;
Ar—O—S(O)₂—, with Ar representing an optionally substituted aryl group; preferentially a phenyl optionally substituted by one or more alkyl groups;
R₁₉ and R₂₀ together form either a double bond or an optionally substituted benzo group D′;
R′₁₆, R′₁₉ and R′₂₀, which may be identical or different, represent a hydrogen atom, a (C₁-C₆)alkyl group or a hydroxyl group;
R₂₁ represents a hydrogen atom, a (C₁-C₆)alkyl group or a (C₁-C₆)alkoxy group;
Rₐ and R_{b}, which may be identical or different, are as defined above; preferentially, Rₐ represents a hydrogen atom and R_{b} represents an aryl group, such as phenyl;
Y represents either a hydroxyl group or an oxo group;
[symbol] represents a single bond when Y is an oxo group, and represents a double bond when Y represents a hydroxyl group;
   it being understood that the formulae (IV) and (IV′) comprise, on one of the rings D or E:
at least one (O)₂S(O⁻)—, M′⁺ group, with M′⁺ representing a cationic counterion; or
at least one (O)CO⁻—, M′⁺ group, with M′⁺ representing a cationic counterion;
preferably at least one sodium sulfonate group.

Mention may be made, as examples of dyes of formula (IV), of: Acid Red 195, Acid Yellow 23, Acid Yellow 27 or Acid Yellow 76, and mention may be made, as examples of dyes of formula (IV′), of: Acid Yellow 17.
c) the anthraquinone dyes of formula (V) or (V′)

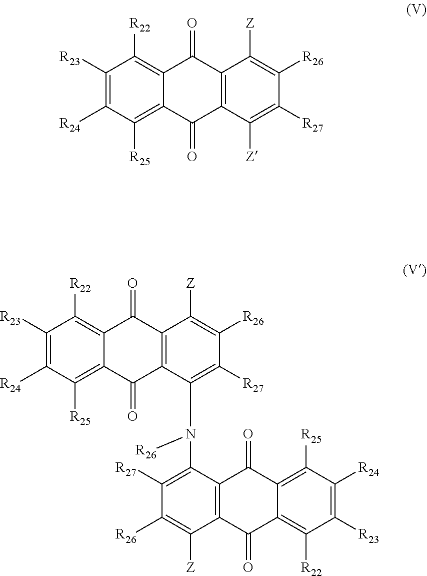

in which formulae (V) and (V′):
R₂₂, R₂₃, R₂₄, R₂₅, R₂₆ and R₂₇, which may be identical or different, represent a hydrogen atom, a halogen atom or a group chosen from:
(C₁-C₆)alkyl;
hydroxyl, mercapto;
(C₁-C₆)alkoxy, (C₁-C₆)alkylthio;
optionally substituted aryloxy or arylthio, preferentially substituted by one or more groups chosen from alkyl and (O)₂S(O⁻)—, M⁺, with M⁺ as defined above;
aryl(C₁-C₆)(alkyl)amino optionally substituted by one or more groups chosen from alkyl and (O)₂S(O⁻)—, M⁺, with M⁺ as defined above;
(di)(C₁-C₆)(alkyl)amino;
(di)(hydroxy(C₁-C₆)alkyl)amino;
(O)₂S(O⁻)—, M⁺, with M⁺ as defined above;
Z′ represents a hydrogen atom or an NR₂₈R₂₉ group with R₂₈ and R₂₉, which may be identical or different, representing a hydrogen atom or a group chosen from:
(C₁-C₆)alkyl;
polyhydroxy(C₁-C₆)alkyl, such as hydroxyethyl;
aryl optionally substituted by one or more groups, particularly i) (C₁-C₆)alkyl, such as methyl, n-dodecyl or n-butyl; ii) (O)₂S(O⁻)—, M⁺, with M⁺ as defined above; iii) R^{o}—C(X)—X′—, R^{o}—X′—C(X)— or R^{o}—X′—C(X)—X″—, with R^{o}, X, X′ and X″ as defined above; preferentially, R^{o} represents a (C₁-C₆)alkyl group;
cycloalkyl, in particular cyclohexyl;
Z represents a group chosen from hydroxyl and NR′₂₈R′₂₉ with R′₂₈ and R′₂₉, which may be identical or different, representing the same atoms or groups as R₂₈ and R₂₉ as defined above;
   it being understood that the formulae (V) and (V′) comprise:
at least one (O)₂S(O⁻)—, M′⁺ group, with M′⁺ representing a cationic counterion; or
at least one (O)CO⁻—, M′⁺ group, with M′⁺ representing a cationic counterion;
   preferably at least one sodium sulfonate group.

Mention my be made, as examples of dyes of formula (V), of: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3 or Ext. Violet No. 2;

and mention may be made, as examples of dyes of formula (V′), of: Acid Black 48.
d) the nitro dyes of formula (VI) or (VI′):

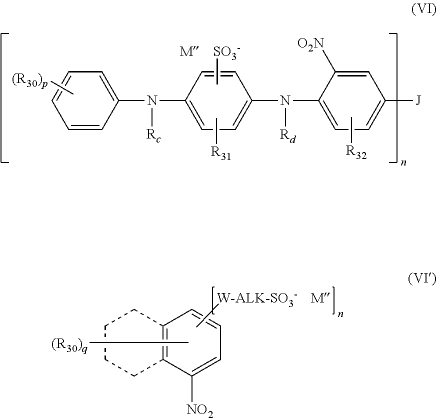

in which formulae (VI) and (VI′):
R₃₀, R₃₁ and R₃₂, which may be identical or different, represent a hydrogen atom, a halogen atom or a group chosen from:
(C₁-C₆)alkyl;
(C₁-C₆)alkoxy optionally substituted by one or more hydroxyl groups or (C₁-C₆)alkylthio optionally substituted by one or more hydroxyl groups;
hydroxyl, mercapto;
nitro, nitroso;
polyhalo(C₁-C₆)alkyl;
R^{o}—C(X)—X′—, R^{o}—X′—C(X)— or R^{o}—X′—C(X)—X″—, with R^{o}, X, X′ and X″ as defined above;
(O)₂S(O⁻)—, M⁺, with M⁺ as defined above;
(O)CO⁻—, M⁺, with M⁺ as defined above;
(di)(C₁-C₆)(alkyl)amino;
(di)(hydroxy(C₁-C₆)alkyl)amino;
heterocycloalkyl, such as piperidino, piperazino or morpholino;
   in particular, R₃₀, R₃₁ and R₃₂ represent a hydrogen atom;
R_{c} and R_{d}, which may be identical or different, represent a hydrogen atom or a (C₁-C₆)alkyl group;
W is as defined above; W represents in particular an —N(H)— group;
ALK represents a linear or branched divalent C₁-C₆ alkylene group; in particular, ALK represents a —CH₂—CH₂— group;
n has a value 1 or 2;
p represents an integer ranging from 1 to 5;
q represents an integer ranging from 1 to 4;
u has a value of 0 or 1;
when n has a value of 1, J represents a nitro or nitroso group; in particular a nitro group;
when n has a value of 2, J represents an oxygen or sulfur atom or a divalent —S(O)ₘ— group with m representing an integer which is 1 or 2; preferentially, J represents an —SO₂— group;
M″ represents a hydrogen atom or a cationic counterion;

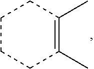

which is present or absent, represents a benzo group optionally substituted by one or more R₃₀ groups as defined above;
it being understood that the formulae (VI) and (VI′) comprise:
at least one (O)₂S(O⁻)—, M′⁺ group, with M′⁺ representing a cationic counterion; or
at least one (O)CO⁻—, M′⁺ group, with M′⁺ representing a cationic counterion;
preferably at least one sodium sulfonate group.

Mention may be made, as examples of dyes of formula (VI), of: Acid Brown 13 and Acid Orange 3; mention may be made, as examples of dyes of formula (VI′), of: Acid Yellow 1, sodium salt of 2,4-dinitro-1-naphthol-7-sulfonic acid, 2-piperidino-5-nitrobenzenesulfonic acid, 2-(4′-N,N-(2″-hydroxyethyl)amino-2′-nitro)anilineethanesulfonic acid, 4-p-hydroxyethylamino-3-nitrobenzenesulfonic acid and Ext. D&C Yellow 7.
e) the triarylmethane dyes of formula (VII):

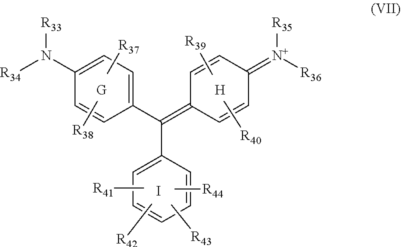

in which formula (VII):
R₃₃, R₃₄, R₃₅ and R₃₆, which may be identical or different, represent a hydrogen atom or a group chosen from (C₁-C₆)alkyl, optionally substituted aryl and optionally substituted aryl(C₁-C₆)alkyl; particularly a (C₁-C₆)alkyl group and benzyl group optionally substituted by an (O)ₘS(O⁻)—, M⁺ group, with M⁺ and m as defined above;
R₃, R₃₈, R₃₉, R₄₀, R₄₁, R₄₂, R₄₃ and R₄₄, which may be identical or different, represent a hydrogen atom or a group chosen from:
(C₁-C₆)alkyl;
(C₁-C₆)alkoxy, (C₁-C₆)alkylthio;
(di)(C₁-C₆)(alkyl)amino;
hydroxyl, mercapto;
nitro, nitroso;
R^{o}—C(X)—X′—, R^{o}—X′—C(X)— or R^{o}—X′—C(X)—X″—, with R^{o} representing a hydrogen atom or an alkyl or aryl group. and X, X′ and X″, which may be identical or different, representing an oxygen or sulfur atom or NR, with R representing a hydrogen atom or a (C₁-C₆)alkyl group;
(O)₂S(O⁻)—, M⁺, with M⁺ representing a hydrogen atom or a cationic counterion;
(O)CO⁻—, M⁺, with M⁺ as defined above;
or else two contiguous groups, R₄₁ with R₄₂ or R₄₂ with R₄₃ or R₄₃ with R₄₄, together form a fused benzo group optionally substituted by one or more groups chosen from i) nitro;
ii) nitroso; iii) (O)₂S(O⁻)—, M⁺; iv) hydroxyl; v) mercapto; vi) (di)(C₁-C₆)(alkyl)amino; vii) R^{o}—C(X)—X′—; viii) R^{o}—X′—C(X)—; ix) R^{o}—X′—C(X)—X″—; with M⁺, R^{o}, X, X′ and X″ as defined above;
   in particular, R₃₇ to R₄₀ represent a hydrogen atom and R₄₁ to R₄₄, which may be identical or different, represent a hydroxyl or (O)₂S(O⁻)—, M⁺ group with M⁺ as defined above; and, when R₄₃ with R₄₄ together form a benzo group, it is preferentially substituted by an (O)₂S(O⁻)— group;
   it being understood that at least one of the rings G, H or I comprises:
at least one (O)₂S(O⁻)—, M′⁺ group, with M′⁺ representing a cationic counterion; or
at least one (O)CO⁻—, M′⁺ group, with M′⁺ representing a cationic counterion;
preferably at least one sodium sulfonate group.

Mention may be made, as examples of dyes of formula (VII), of: Acid Blue 1, Acid Blue 3, Acid Blue 7, Acid Blue 9, Acid Violet 49, Acid Green 3, Acid Green 5 and Acid Green 50.
f) the xanthene-based dyes of formula (VIII):

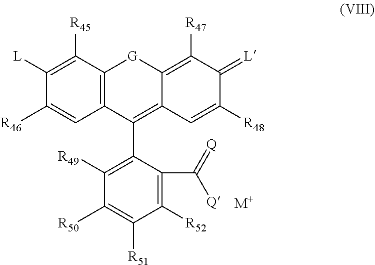

in which formula (VIII):
R₄₅, R₄₆, R₄₇ and R₄₈, which may be identical or different, represent a hydrogen atom or a halogen atom;
R₄₉, R₅₀, R₅₁ and R₅₂, which may be identical or different, represent a hydrogen atom, a halogen atom or a group chosen from:
(C₁-C₆)alkyl;
(C₁-C₆)alkoxy, (C₁-C₆)alkylthio;
hydroxyl, mercapto;
nitro, nitroso;
(O)₂S(O⁻)—, M⁺, with M⁺ representing a hydrogen atom or a cationic counterion;
(O)CO⁻—, M⁺, with M⁺ as defined above;
in particular, R₅₃, R₅₄, R₅₅ and R₄₈ represent a hydrogen atom or a halogen atom;
G represents an oxygen or sulfur atom or an NR_{c} group, with R_{c} as defined above; in particular, G represents an oxygen atom;
L represents an alkoxide O—, M⁺, a thioalkoxide S—, M⁺, or an NR_{f} group, with R, representing a hydrogen atom or a (C₁-C₆)alkyl group, and M⁺ as defined above; M⁺ is in particular sodium or potassium;
L′ represents an oxygen or sulfur atom or an ammonium group: N⁺R_{f}R_{g}, with R_{f} and R_{g}, which may be identical or different, representing a hydrogen atom, a (C₁-C₆)alkyl group or an optionally substituted aryl group; L′ particularly represents an oxygen atom or a phenylamino group optionally substituted with one or more alkyl or (O)ₘS(O⁻)—, M⁺ groups with m and M⁺ as defined above;
Q and Q′, which may be identical or different, represent an oxygen or sulfur atom; in particular, Q and Q′ represent an oxygen atom;
M⁺ is as defined above.

Mention may be made, as examples of dyes of formula (VIII), of: Acid Yellow 73, Acid Red 51, Acid Red 52, Acid Red 87, Acid Red 92, Acid Red 95 and Acid Violet 9.
g) the indole-based dyes of formula (IX):

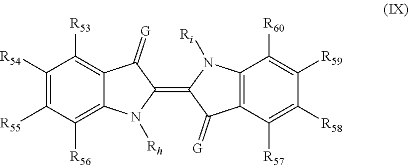

in which formula (IX):
R₅₃, R₅₄, R₅₅, R₅₆, R₅₇, R₅₈, R₅₉ and R₆₀, which may be identical or different, represent a hydrogen atom or a group chosen from:
(C₁-C₆)alkyl;
(C₁-C₆)alkoxy, (C₁-C₆)alkylthio;
hydroxyl, mercapto;
nitro, nitroso;
R^{o}—C(X)—X′—, R^{o}—X′—C(X)— or R^{o}—X′—C(X)—X″—, with R^{o} representing a hydrogen atom or an alkyl or aryl group. and X, X′ and X″, which may be identical or different, representing an oxygen or sulfur atom or NR, with R representing a hydrogen atom or a (C₁-C₆)alkyl group;
(O)₂S(O⁻)—, M⁺, with M⁺ representing a hydrogen atom or a cationic counterion;
(O)CO⁻—, M⁺, with M⁺ as defined above;
G represents an oxygen or sulfur atom or an NR_{c} group, with R_{c} as defined above; in particular, G represents an oxygen atom;
Rᵢ and Rₕ, which may be identical or different, represent a hydrogen atom or a (C₁-C₆)alkyl group;
   it being understood that the formula (IX) comprises:
at least one (O)₂S(O⁻)—, M′⁺ group, with M′⁺ representing a cationic counterion; or
at least one (O)CO⁻—, M′⁺ group, with M′⁺ representing a cationic counterion;
   preferably at least one sodium sulfonate group.

Mention may be made, as example of dyes of formula (IX), of: Acid Blue 74.
h) the quinoline-based dyes of formula (X):

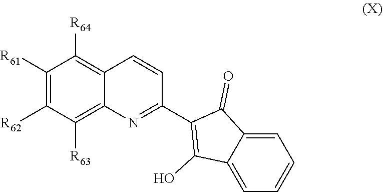

in which formula (X):
R₆₁ represents a hydrogen or halogen atom or a (C₁-C₆)alkyl group;
R₆₂, R₆₃ and R₆₄, which may be identical or different, represent a hydrogen atom or an (O)₂S(O⁻)—, M⁺ group, with M⁺ representing a hydrogen atom or a cationic counterion; or else R₆₁ with R₆₂ or R₆₁ with R₆₄ together form a benzo group optionally substituted by one or more (O)₂S(O⁻)—, M⁺ groups, with M⁺ representing a hydrogen atom or a cationic counterion;
it being understood that the formula (X) comprises at least one (O)₂S(O⁻)—, M′⁺ group, with M′⁺ representing a cationic counterion, preferably at least one sodium sulfonate group.

Mention may be made, as examples of dyes of formula (X), of: Acid Yellow 2, Acid Yellow 3 and Acid Yellow 5.

More particularly, the dyes of formulae (III) to (VIII) that are useful in the invention are chosen from: Acid Red 87 (VIII) (C.I. 45380); sodium salt of 2,4-dinitro-1-naphthol-7-sulfonic acid (VI′) (C.I. 10316); Acid Orange 3 (VI) (C.I. 10383); Acid Yellow 9/Food Yellow 2 (III) (C.I. 13015); Direct Red 45/Food Red 13(1l) (C.I. 14780); Acid Black 52 (III) (C.I. 13711); Acid Yellow 36 (III) (C.I. 13065); sodium salt of 1-hydroxy-2-(2′,4′-xylyl-5-sulfonatoazo)naphthalene-4-sulfonic acid/Food Red 1 (III) (C.I. 14700); Acid Red 14/Food Red 3/Mordant Blue 79 (III) (C.I. 14720); sodium salt of 4-hydroxy-3-[(2-methoxy-5-nitrophenyl)diaza]-6-(phenylamino)naphthalene-2-sulfonic acid/Acid Brown 4 (III) (C. I. 14805); Acid Orange 7/Pigment Orange 17/Solvent Orange 49 (III) (C.I. 15510); Food Yellow 3/Pigment Yellow 104 (III) (C.I. 15985); Acid Red 27/Food Red 9 (III) (C.I. 16185); Acid Orange 10/Food Orange 4 (III) (C.I. 16230); Acid Red 44 (III) (C.I. 16250); Acid Red 33/Food Red 12 (III) (C.I. 17200); Acid Red 184 (III) (C.I. 15685); Acid Violet 3 (III) (C.I. 19125); sodium salt of 1-hydroxy-2-(4′-acetamidophenylazo)-8-acetamidonaphthalene-3,6-disulfonic acid/Acid Violet 7/Food Red 11 (III) (C.I. 18055); Acid Red 135 (III) (C.I. 18130); Acid Yellow 27 (IV) (C.I. 19130); Acid Yellow 23/Food Yellow 4 (IV) (C.I. 19140); 4′-(sulfonato-2″,4″-dimethyl)bis(2,6-phenylazo)-1.3-dihydroxybenzene/Acid Orange 24 (III) (C.I. 20170); sodium salt of 1-amino-2-(4′-nitrophenylazo)-7-phenylazo-8-hydroxynaphthalene-3,6-disulfonic acid/Acid Black 1 (III) (C.I. 20470); (4-((4-methylphenyl)sulfonyloxy)phenylazo)-2,2′-dimethyl-4-((2-hydroxy-5,8-disulfonato)naphthylazo)biphenyl/Acid Red 111 (III′) (C.I. 23266); Food Black 2 (III) (C.I. 27755); 1-(4′-sulfonatophenylazo)-4-((2″-hydroxy-3″-acetylamino-6″,8″-disulfonato)naphthylazo)-6-sulfonatonaphthalene (tetrasodium salt)/Food Black 1 (III) (C.I. 25440); Acid Blue 9 (VII) (C.I. 42090); Acid Violet 43 (V) (C.I. 60730); Acid Green 25 (V) (C.I. 61570); sodium salt of 1-amino-4-cyclohexylamino-9,10-anthraquinone-2-sulfonic acid/Acid Blue 62 (V) (C.I. 62045); Acid Blue 78 (V) (C.I. 62105); sodium salt of 4-hydroxy-3-((2-methoxyphenyl)azo)-1-naphthalenesulfonic acid/Acid Red 4 (III) (C.I. 14710); 2-piperidino-5-nitrobenzenesulfonic acid (VI′); 2-(4′-N,N-(2″-hydroxyethyl)amino-2′-nitro)anilineethanesulfonic acid (VI′); 4-β-hydroxyethylamino-3-nitrobenzenesulfonic acid (VI′); Acid Violet 49 (VII) (C.I. 42640); Acid Blue 7 (VII) (C.I. 42080); sodium salt of 1,2-dihydroxy-3-sulfoanthraquinone/Mordant Red 3 (V) (C.I. 58005); Sodium salt of 1-amino-9,10-dihydro-9,10-dioxo-4-(phenylamino)-2-anthracenesulfonic acid/Acid Blue 25 (V) (C.I. 62055); sodium salt of 4-hydroxy-3-((2-methoxyphenyl)azo)-1-naphthalenesulfonic acid/Acid Red 4 (III) (C.I. 14710).

Most of these dyes are described in particular in the Colour Index published by The Society of Dyers and Colourists, P.O. Box 244, Perkin House, 82 Grattan Road, Bradford, Yorkshire, BD1 2JBN, England.

The anionic dyes which are most particularly preferred are the dyes designated in the Colour Index under the code C.I. 58005 (monosodium salt of 1,2-dihydroxy-9,10-anthraquinone-3-sulfonic acid), C.I. 60730 (monosodium salt of 2-[(9,10-dihydro-4-hydroxy-9,10-dioxo-1-anthracenyl)amino]-5-methylbenzenesulfonic acid), C.I. 15510 (monosodium salt of 4-[(2-hydroxy-1-naphthalenyl)azo]benzenesulfonic acid), C.I. 15985 (disodium salt of 6-hydroxy-5-[(4-sulfophenyl)azo]-2-naphthalenesulfonic acid), C.I. 17200 (disodium salt of 5-amino-4-hydroxy-3-(phenylazo)-2,7-naphthalenedisulfonic acid), C.I. 20470 (disodium salt of 1-amino-2-(4′-nitrophenylazo)-7-phenylazo-8-hydroxy-3,6-naphthalenedisulfonic acid), C.I. 42090 (disodium salt of N-ethyl-N-[4-[[4-[ethyl[(3-sulfophenyl)methyl]amino]phenyl](2-sulfophenyl)methylene]-2,5-cyclohexadien-1-ylidene]-3-sulfobenzenemethanaminium hydroxide, internal salt), C.I. 61570 (disodium salt of 2,2′-[(9,10-dihydro-9,10-dioxo-1,4-anthracenediyl)diimino]bis[5-methylbenzenesulfonic acid]).

Use may also be made of the compounds corresponding to the mesomeric or tautomeric forms of the structures (III) to (X).

### Natural Dyes

The direct dyes may be chosen from natural direct dyes.

Mention may be made, among the natural direct dyes which can be used according to the invention, of lawsone, juglone, alizarin, purpurin, carminic acid, kermesic acid, purpurogallin, protocatechaldehyde, indigo, isatin, curcumin, spinulosin, apigenidin, orceins, brazilin, brazilein, haematein or hematoxylin. Use may also be made of extracts or decoctions containing these natural dyes and in particular henna-based poultices or extracts.

According to a preferred embodiment, the direct dyes are chosen from the triarylmethane direct dyes of following formulae (IIa₁) and (IIa₂) and their mixtures:

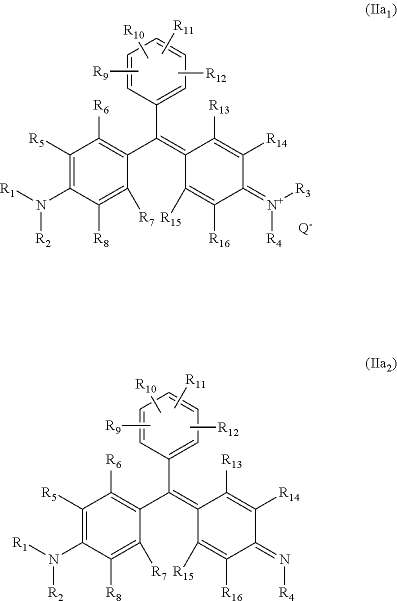

in which:
R₁, R₂, R₃ and R₄, which may be identical or different, represent a hydrogen atom or a group from among: (C₁-C₆)alkyl which is optionally substituted, preferably by a hydroxyl group; aryl, such as phenyl, aryl(C₁-C₄)alkyl, such as benzyl, heteroaryl or heteroaryl(C₁-C₄)alkyl, or else two R₁ and R₂ and/or R₃ and R₄ groups, borne by the same nitrogen atom, form, together with the nitrogen atom which bears them, an optionally substituted heterocycloalkyl group, such as morpholino, piperazino or piperidino; preferably, R₁, R₂, R₃ and R₄, which may be identical or different, represent a hydrogen atom or a (C₁-C₄)alkyl group;
R₅, R₆, R₇, R₈, R₉, R₁₀, R₁₁, R₁₂, R₁₃, R₁₄, R₁₅, and R₁₆, which may be identical or different, represent a hydrogen atom, a halogen atom or a group chosen from i) hydroxyl, ii) thiol, iii) amino, iv) (di)(C₁-C₄)(alkyl)amino, v) (di)arylamino, such as (di)phenylamino, vi) nitro, vii) acylamino (—NR—C(O)R′), in which the R radical is a hydrogen atom or a C₁-C₄ alkyl radical optionally bearing at least one hydroxyl group and the R′ radical is a C₁-C₂ alkyl radical; viii) carbamoyl ((R)₂N—C(O)—), in which the R radicals, which may be identical or different, represent a hydrogen atom or a C₁-C₄ alkyl radical optionally bearing at least one hydroxyl group; ix) carboxylic acid or ester (—O—C(O)R′) or (—C(O)OR′), in which the R′ radical is a hydrogen atom or C₁-C₄ alkyl optionally bearing at least one hydroxyl group and the R′ radical is a C₁-C₂ alkyl radical; x) alkyl which is optionally substituted, in particular by a hydroxyl group; xi) alkylsulfonylamino (R′SO₂—NR—), in which the R radical represents a hydrogen atom or a C₁-C₄ alkyl radical optionally bearing at least one hydroxyl group and the R′ radical represents a C₁-C₄ alkyl radical, a phenyl radical; xii) aminosulfonyl ((R)₂N—SO₂—), in which the R radicals, which may be identical or different, represent a hydrogen atom or a C₁-C₄ alkyl radical optionally bearing at least one hydroxyl group; xiii) (C₁-C₄)alkoxy; and xiv) (C₁-C₄)alkylthio;
or else two radicals borne by two contiguous carbon atoms, R₅ and R₆ and/or R₇ and R₈ and/or R₉ and R₁₀ and/or R₁₁ and R₁₂ and/or R₁₃ and R₁₄ and/or R₁₅ and R₁₆, form, together with the carbon atoms which bear them, a fused 6-membered aryl or heteroaryl, preferably benzo, ring, it being possible for said ring in addition to optionally be substituted, preferably an unsubstituted benzo ring;
Q⁻ represents an anionic counterion for achieving electrical neutrality, preferably chosen from halides, such as chloride or bromide, and phosphate.

According to this preferred embodiment, the direct dye is preferably HC Blue 15.

According to a variant of the invention, the direct dye(s) are chosen from cationic direct dyes, preferably endocyclic cationic direct dyes, more particularly of formula (IV-1) as defined above, such as for example Basic Red 51.

The direct dye(s) are preferably chosen from cationic direct dyes, more preferentially from the triarylmethane direct dyes of formulae (IIa₁) and (IIa₂) as defined above, the direct dyes of formula (IV-1) as defined above, and mixtures thereof, even more preferentially from HC Blue 15, Basic Red 51, and mixtures thereof.

The direct dye(s) can be present in the composition in a total content ranging from 0.001% to 5% by weight, preferably from 0.01% to 3% by weight, more preferentially from 0.1% to 1% by weight, even more preferentially from 0.1% to 0.8% by weight, relative to the total weight of the composition.

According to a preferred embodiment, the composition comprises a total content of colouring agents of less than 0.1% by weight, preferably of less than 0.01% by weight, more preferentially of less than 0.001% by weight, relative to the total weight of the composition.

According to a more preferred embodiment, the composition is devoid of colouring agents.

### Additional Basifying Agents

The composition can also comprise one or more additional basifying agents other than the carbonates, bicarbonates and silicates as defined above.

The additional basifying agent(s) can be inorganic or organic. They can be chosen from i) aqueous ammonia, ii) alkanolamines, such as monoethanolamine, diethanolamine, triethanolamine and also their derivatives, iii) oxyethylenated and/or oxypropylenated ethylenediamines, iv) inorganic or organic hydroxides, v) amino acids, preferably basic amino acids, such as arginine, lysine, ornithine, citrulline and histidine, and vi) the compounds of following formula (II):

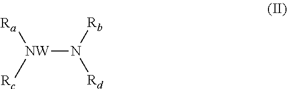

in which:
W is a divalent (C₁-C₈)alkylene group, preferably a propylene group, optionally substituted in particular by a hydroxyl group or a C₁-C₄ alkyl radical;
Rₐ, R_{b}, R_{c} and R_{d}, which may be identical or different, represent a hydrogen atom or a C₁-C₄ alkyl or C₁-C₄ hydroxyalkyl radical;
vii) and mixtures thereof.

The inorganic or organic hydroxides are preferably chosen from i) alkali metal hydroxides, such as sodium hydroxide or potassium hydroxide, ii) alkaline earth metal hydroxides, iii) transition metal hydroxides, such as hydroxides of metals from Groups III, IV, V and VI, iv) hydroxides of lanthanides or of actinides.

When they are present, the additional basifying agent(s) preferably represent from 0.001% to 20% by weight, more particularly from 0.005% to 16% by weight, relative to the total weight of the composition.

According to a preferred embodiment, the composition according to the invention does not comprise an additional basifying agent chosen from aqueous ammonia and/or alkanolamines.

### Acidifyinq Agents

The composition can additionally comprise one or more acidifying agents.

Mention may be made, among the acidifying agents, by way of example, of inorganic acids, for instance hydrochloric acid, (ortho)phosphoric acid, boric acid, nitric acid or sulfuric acid, or organic acids, for instance compounds comprising at least one carboxylic acid functional group, such as acetic acid, tartaric acid, citric acid or lactic acid, one sulfonic acid functional group, one phosphonic acid functional group or one phosphoric acid functional group.

### pH of the Composition

The composition according to the invention preferably exhibits a pH of less than or equal to 11, preferably of less than or equal to 10.5, preferably of less than or equal to 10.

The pH of the composition according to the invention may vary from 8 to 11, preferably from 8 to 10.5, more preferentially from 8 to 10.

According to a particularly preferred embodiment, the pH of the composition according to the invention varies from 8.3 to 10.

### Sequestrants

The composition according to the invention optionally contains one or more sequestrants. As examples of sequestrants that may be used in the present invention, mention may be made of N,N-dicarboxymethyl-L-glutamic acid and tetrasodium N,N-bis(carboxymethyl)-L-glutamate.

### Other Characteristics of the Composition

The composition preferably comprises water in a content ranging from 5% to 99% by weight, more preferentially ranging from 5% to 80% by weight, relative to the total weight of the composition.

The composition can additionally comprise at least one organic solvent.

The term “organic solvent” means an organic substance which is capable of dissolving another substance without chemically modifying it.

Mention may be made, as organic solvent, for example, of lower C₂-C₄ alkanols, such as ethanol and isopropanol; polyols and polyol ethers, such as 2-butoxyethanol, propylene glycol, propylene glycol monomethyl ether, diethylene glycol monoethyl ether or diethylene glycol monomethyl ether, and also aromatic alcohols, such as benzyl alcohol or phenoxyethanol, and mixtures thereof.

The organic solvents are present in proportions preferably ranging from 0.1% to 40% by weight, more preferentially from 1% to 30% by weight, even more preferentially from 1% to 25% by weight, relative to the total weight of the composition.

The composition according to the invention can be provided in liquid form, in the form of a serum, in thickened form, in particular a gel, a cream, a wax or a paste, or in foam form.

The composition according to the invention can also comprise one or more additional compounds chosen from nonionic, anionic, cationic or amphoteric surfactants, cationic, anionic, nonionic or zwitterionic, associative or non-associative, thickening polymers of natural or synthetic origin, silicones in the form of oils, gums or resins or non-silicone plant, mineral or synthetic oils, UV-screening agents, fillers, such as pearlescent agents and metal oxides, such as titanium dioxides, clays, fragrances, peptizing agents, vitamins and preservatives.

### Process for Lightening Keratin Fibres

According to a second aspect, a subject of the present invention is a process for lightening keratin fibres, comprising the application to the keratin fibres of a composition comprising ingredients i) to iv) as defined above.

### Process for Simultaneously Bleaching and Dyeing Keratin Fibres

According to a third aspect, a subject of the present invention is a process for simultaneously bleaching and dyeing keratin fibres, comprising the application to the keratin fibres of a composition comprising ingredients i) to iv) as defined above and v) one or more colouring agents chosen from direct dyes, oxidation dyes, and mixtures thereof.

### Additional Characteristics of the Process for Lightening or Simultaneously Bleaching and Dyeing Keratin Fibres

In particular, the composition is applied to wet or dry keratin fibres.

Preferably, the keratin fibres are dark keratin fibres.

The term “dark keratin fibres” means keratin fibres with a tone depth of less than or equal to 6 (dark blond) and preferably less than or equal to 4 (chestnut-brown).

The composition may advantageously be applied to the keratin fibres in an amount ranging from 0.1 g to 20 g of composition per gram of keratin fibres.

The composition is left to stand on the fibres for a period generally of from 1 minute to 1 hour, preferably from 5 minutes to 60 minutes.

By way of example, the composition may be left to stand on the fibres for a period of 50 minutes.

The composition may be left to stand on the fibres under an occlusive system. A non-limiting example of an occlusive system that may be mentioned is an occlusive system of envelope type made of aluminium or plastic film or of hair cap type with or without holes.

The temperature during the lightening process is conventionally between ambient temperature (between 15° C. and 25° C.) and 80° C., and preferably between ambient temperature and 60° C.

By way of example, the temperature during the lightening process is 33° C.

On conclusion of the treatment, the keratin fibres are optionally rinsed with water, optionally washed with a shampoo and then rinsed with water, before being dried or left to dry naturally.

The drying step may be performed using absorbent paper, a hairdryer or a styling hood.

The composition used in the process according to the present invention is preferably prepared by mixing at least two compositions. Preferably, the mixing of said at least two compositions is carried out extemporaneously, before the application of the composition to the keratin fibres.

According to a preferred embodiment, the composition used in the process according to the present invention results from the mixing:
of a composition (A) comprising:
   one or more chemical oxidizing agents chosen from hydrogen peroxide, hydrogen peroxide-generating systems other than peroxygenated salts, and mixtures thereof, as defined above, preferably hydrogen peroxide; and
of a composition (B) comprising:
   one or more compounds chosen from carbonates, carbonate-generating systems, bicarbonates, bicarbonate-generating systems, and mixtures thereof, as defined above, preferably chosen from carbonates, bicarbonates, and mixtures thereof, as defined above; and
   one or more silicates as defined above; and
   optionally one or more colouring agents chosen from direct dyes, oxidation dyes, and mixtures thereof; and
   optionally one or more compounds chosen from esters, amides, imines, and mixtures thereof, as defined above; and
optionally of a composition (C) comprising:
   one or more colouring agents chosen from direct dyes, oxidation dyes, and mixtures thereof; and
   optionally one or more compounds chosen from esters, amides, imines, and mixtures thereof, as defined above;
      it being understood that composition (B) or at least one of compositions (B) and (C), when composition (C) is present, comprises one or more compounds chosen from esters, amides, imines, and mixtures thereof, as defined above.

In the case where the process according to the present invention is a process for lightening keratin fibres, the composition used in this process preferably results from the mixing:
of a composition (A) comprising:
   one or more chemical oxidizing agents chosen from hydrogen peroxide, hydrogen peroxide-generating systems other than peroxygenated salts, and mixtures thereof, as defined above, preferably hydrogen peroxide; and
of a composition (B) comprising:
   one or more compounds chosen from carbonates, carbonate-generating systems, bicarbonates, bicarbonate-generating systems, and mixtures thereof, as defined above, preferably chosen from carbonates, bicarbonates, and mixtures thereof, as defined above; and
   one or more silicates as defined above; and
   one or more compounds chosen from esters, amides, imines, and mixtures thereof, as defined above.

In the case where the process according to the present invention is a process for simultaneously bleaching and dyeing keratin fibres, the composition used in this process may result from the mixing:
of a composition (A) comprising:
   one or more chemical oxidizing agents chosen from hydrogen peroxide, hydrogen peroxide-generating systems other than peroxygenated salts, and mixtures thereof, as defined above, preferably hydrogen peroxide; and
of a composition (B) comprising:
   one or more compounds chosen from carbonates, carbonate-generating systems, bicarbonates, bicarbonate-generating systems, and mixtures thereof, as defined above, preferably chosen from carbonates, bicarbonates, and mixtures thereof, as defined above; and
   one or more silicates as defined above; and
   optionally one or more colouring agents chosen from direct dyes, oxidation dyes, and mixtures thereof; and
   one or more compounds chosen from esters, amides, imines, and mixtures thereof, as defined above.

In the case where the process according to the present invention is a process for simultaneously bleaching and dyeing keratin fibres, the composition used in this process may result from the mixing:
of a composition (A) comprising:
   one or more chemical oxidizing agents chosen from hydrogen peroxide, hydrogen peroxide-generating systems other than peroxygenated salts, and mixtures thereof, as defined above, preferably hydrogen peroxide; and
of a composition (B) comprising:
   one or more compounds chosen from carbonates, carbonate-generating systems, bicarbonates, bicarbonate-generating systems, and mixtures thereof, as defined above, preferably chosen from carbonates, bicarbonates, and mixtures thereof, as defined above; and
   one or more silicates as defined above; and
   optionally one or more colouring agents chosen from direct dyes, oxidation dyes, and mixtures thereof; and
   optionally one or more compounds chosen from esters, amides, imines, and mixtures thereof, as defined above; and
of a composition (C) comprising:
   one or more colouring agents chosen from direct dyes, oxidation dyes, and mixtures thereof; and
   optionally one or more compounds chosen from esters, amides, imines, and mixtures thereof, as defined above;
      it being understood that at least one of compositions (B) and (C) comprises one or more compounds chosen from esters, amides, imines, and mixtures thereof, as defined above.

Preferably, when the composition according to the invention results from the mixing of the compositions (A), (B) and (C) as defined above, composition (B) does not comprise colouring agent(s) chosen from direct dyes, oxidation dyes, and mixtures thereof.

Preferably, at least one of the compositions (A) or (B) or at least one of the three compositions (A) or (B) or (C) is aqueous. More preferentially, the composition (A) is aqueous.

According to a particular embodiment, the composition (B) is anhydrous.

According to a particular embodiment, the composition (A) is aqueous, the composition (B) is anhydrous and the composition (C), when it is present, is aqueous.

The term “aqueous composition” means a composition comprising at least 2% by weight of water, preferably at least 5% by weight of water, more preferentially at least 10% by weight of water, and even more advantageously more than 20% by weight of water.

The term “anhydrous composition” means a composition containing less than 2% by weight of water, preferably less than 0.5% by weight of water, more preferentially devoid of water. If applicable, such small amounts of water may in particular be introduced by ingredients of the composition which may contain residual amounts thereof.

The composition used in the process according to the present invention makes it possible to obtain a lightening of keratin fibres characterized by a value of b* which is lower, preferably 10% lower, more preferentially 15% lower, than the value of b* measured at the same intensity level L* on keratin fibres that have been lightened using a composition comprising one or more persulfates, the values of b* and L* being measured in the CIE L*a*b* system.

Preferably, the values of b* and L* are measured according to the colour evaluation method described in the examples.

### Uses

According to a fourth aspect, a subject of the present invention is the use of a composition comprising ingredients i) to iv) as defined above for lightening keratin fibres, preferably for lightening keratin fibres while at the same time making them less yellow.

According to a fifth aspect, a subject of the present invention is the use of a composition comprising ingredients i) to iv) as defined above and v) one or more colouring agents chosen from direct dyes, oxidation dyes, and mixtures thereof, for the simultaneous bleaching and dyeing of keratin fibres.

### Multi-Compartment Device (Kit)

According to a sixth aspect, a subject of the present invention is a device having multiple separate compartments (kit), comprising:
a first compartment containing a composition (A) comprising:
   one or more chemical oxidizing agents chosen from hydrogen peroxide, hydrogen peroxide-generating systems other than peroxygenated salts, and mixtures thereof, as defined above, preferably hydrogen peroxide; and
a second compartment containing a composition (B) comprising:
   one or more compounds chosen from carbonates, carbonate-generating systems, bicarbonates, bicarbonate-generating systems, and mixtures thereof, as defined above, preferably chosen from carbonates, bicarbonates, and mixtures thereof, as defined above; and
   one or more silicates as defined above; and
   optionally one or more colouring agents chosen from direct dyes, oxidation dyes, and mixtures thereof; and
   optionally one or more compounds chosen from esters, amides, imines, and mixtures thereof, as defined above; and
optionally a third compartment containing a composition (C) comprising:
   one or more colouring agents chosen from direct dyes, oxidation dyes, and mixtures thereof; and
   optionally one or more compounds chosen from esters, amides, imines, and mixtures thereof, as defined above;
      it being understood that composition (B) or at least one of compositions (B) and (C), when composition (C) is present, comprises one or more compounds chosen from esters, amides, imines, and mixtures thereof, as defined above.

### EXAMPLES

The examples that follow allow the invention to be understood more clearly, without, however, being limiting in nature. In the examples that follow, unless otherwise indicated, all the amounts are shown as weight percentages relative to the total weight of the composition.

### Colour Evaluation Method

In these examples, the colour of the locks was evaluated in the CIE L* a* b* system, using a colorimeter which is a Minolta CM3617A Spectrophotometer (illuminant D65). In this L* a* b* system, L* represents the intensity of the colour, a* indicates the shade of the colour on the green/red colour axis and b* indicates the shade of colour on the blue/yellow colour axis. The higher the value of L*, the lighter the colour. The higher the value of a*, the redder the colour and the higher the value of b*, the yellower the colour.

### Example 1 (Comparative)

The following compositions C1 to C12 were prepared and then applied according to the application protocol described below:

TABLE 1

| Ingredients | C1 | C2 | C3 | C4 | C5 | C6 |
|---|---|---|---|---|---|---|
| Oxidizing | 70.0 | 70.0 | 70.0 | 70.0 | 70.0 | 70.0 |
| cream Blond | (i.e. | (i.e. | (i.e. | (i.e. | (i.e. | (i.e. |
| Studio 40 | 8.4 of | 8.4 of | 8.4 of | 8.4 of | 8.4 of | 8.4 of |
| Vol. | H₂O₂) | H₂O₂) | H₂O₂) | H₂O₂) | H₂O₂) | H₂O₂) |
| L'Oreal | | | | | | |
| Professionnel | | | | | | |
| (12% of | | | | | | |
| H₂O₂) | | | | | | |
| Potassium | 15.0 | 12.5 | 10.0 | 8.0 | 6.0 | 4.0 |
| persulfate | | | | | | |
| Sodium | 10.0 | 10.0 | 10.0 | 8.0 | 8.0 | 8.0 |
| silicate | | | | | | |
| (CAS: | | | | | | |
| 1344-09-8) | | | | | | |
| Water | qs 100 | qs 100 | qs 100 | qs 100 | qs 100 | qs 100 |

TABLE 2

| Ingredients | C7 | C8 | C9 | C10 | C11 | C12 |
|---|---|---|---|---|---|---|
| Oxidizing | 70.0 | 70.0 | 70.0 | 70.0 | 70.0 | 70.0 |
| cream | (i.e. | (i.e. | (i.e. | (i.e. | (i.e. | (i.e. |
| Blond | 8.4 of | 8.4 of | 8.4 of | 8.4 of | 8.4 of | 8.4 of |
| Studio | H₂O₂) | H₂O₂) | H₂O₂) | H₂O₂) | H₂O₂) | H₂O₂) |
| 40 Vol. | | | | | | |
| L'Oreal | | | | | | |
| Professionnel | | | | | | |
| (12% of | | | | | | |
| H₂O₂) | | | | | | |
| Potassium | 8.0 | 6.0 | 4.0 | 8.0 | 6.0 | 4.0 |
| persulfate | | | | | | |
| Sodium | 6.0 | 6.0 | 6.0 | 4.0 | 4.0 | 4.0 |
| silicate | | | | | | |
| (CAS: | | | | | | |
| 1344-09-8) | | | | | | |
| Water | qs 100 | qs 100 | qs 100 | qs 100 | qs 100 | qs 100 |

### Application Protocol

10 g of each of the compositions C₁ to C₁₂ are applied to 12 locks of 1 g of Caucasian HT4 hair on a hot plate maintained at a temperature of 33° C. The whole thing is covered with a cellophane film for 50 min.

The locks are subsequently rinsed, washed with a standard shampoo, rinsed again and then dried.

### Colorimetric Measurements

The results of the colorimetric measurements are summarized in the following table:

TABLE 3

| Composition | L* | b* |
|---|---|---|
| C1 | 57.37 | 30.95 |
| C2 | 56.34 | 31.74 |
| C3 | 54.6 | 30.25 |
| C4 | 54.05 | 30.19 |
| C5 | 50.62 | 28.88 |
| C6 | 48.51 | 29.48 |
| C7 | 49.91 | 29.48 |
| C8 | 47.53 | 27.87 |
| C9 | 46.64 | 26.82 |
| C10 | 47.01 | 27.04 |
| C11 | 45.33 | 26.08 |
| C12 | 41.44 | 23.77 |

The results show that comparative compositions comprising a persulfate make it possible to obtain a good level of lightening characterized by relatively high values of L*, but the colour shades obtained have a pronounced yellow component characterized by high values of b*.

### Example 2

The following compositions C13 to C35 were prepared and then applied according to the application protocol described below:

TABLE 4

| Ingredients | C13 | C14 | C15 | C16 | C17 |
|---|---|---|---|---|---|
| Oxidizing cream | 60.0 | 60.0 | 60.0 | 60.0 | 60.0 |
| Blond Studio 30 Vol. | (i.e. 5.4 | (i.e. 5.4 | (i.e. 5.4 | (i.e. 5.4 | (i.e. 5.4 |
| L'Oréal Professionnel | of | of | of | of | of |
| (9% of H₂O₂) | H₂O₂) | H₂O₂) | H₂O₂) | H₂O₂) | H₂O₂) |
| Sodium silicate | 13.0 | 13.0 | 13.0 | 13.0 | 13.0 |
| (CAS: 1344-09-8) | | | | | |
| Sodium metasilicate | 4.5 | 4.5 | 4.5 | 4.5 | 4.5 |
| (CAS: 6834-92-0) | | | | | |
| Ammonium | 13.33 | 13.33 | 13.33 | 13.33 | 13.33 |
| bicarbonate | | | | | |
| Ethyl linoleate | 9.17 | — | — | — | — |
| (Compound 1) | | | | | |
| Sorbitan oleate | — | 9.17 | — | — | — |
| (Compound 4) | | | | | |
| Polyglyceryl-6 dioleate | — | — | 9.17 | — | — |
| (Compound 5) | | | | | |
| Myristyl stearate | — | — | — | 9.17 | — |
| (Compound 6) | | | | | |
| Oleyl erucate | — | — | — | — | 9.17 |
| (Compound 12) | | | | | |

TABLE 5

| Ingredients | C18 | C19 | C20 | C21 | C22 |
|---|---|---|---|---|---|
| Oxidizing cream | 60.0 | 60.0 | 60.0 | 60.0 | 60.0 |
| Blond Studio 30 Vol. | (i.e. 5.4 | (i.e. 5.4 | (i.e. 5.4 | (i.e. 5.4 | (i.e. 5.4 |
| L'Oréal Professionnel | of | of | of | of | of |
| (9% of H₂O₂) | H₂O₂) | H₂O₂) | H₂O₂) | H₂O₂) | H₂O₂) |
| Sodium silicate | 13.0 | 13.0 | 13.0 | 13.0 | 13.0 |
| (CAS: 1344-09-8) | | | | | |
| Sodium metasilicate | 4.5 | 4.5 | 4.5 | 4.5 | 4.5 |
| (CAS: 6834-92-0) | | | | | |
| Ammonium | 13.33 | 13.33 | 13.33 | 13.33 | 13.33 |
| bicarbonate | | | | | |
| Pentaerythrityl | 9.17 | — | — | — | — |
| tetraisostearate | | | | | |
| (Compound 15) | | | | | |
| Hexyl laurate | — | 9.17 | — | — | — |
| (Compound 16) | | | | | |
| Sorbitan trioleate | — | — | 9.17 | — | — |
| (Compound 22) | | | | | |
| Isostearyl isostearate | — | — | — | 9.17 | — |
| (Compound 24) | | | | | |
| Isostearyl | — | — | — | — | 9.17 |
| neopentanoate | | | | | |
| (Compound 26) | | | | | |

TABLE 6

| Ingredients | C23 | C24 | C25 | C26 | C27 |
|---|---|---|---|---|---|
| Oxidizing cream | 60.0 | 60.0 | 60.0 | 60.0 | 60.0 |
| Blond Studio 30 Vol. | (i.e. 5.4 | (i.e. 5.4 | (i.e. 5.4 | (i.e. 5.4 | (i.e. 5.4 |
| L'Oréal Professionnel | of | of | of | of | of |
| (9% of H₂O₂) | H₂O₂) | H₂O₂) | H₂O₂) | H₂O₂) | H₂O₂) |
| Sodium silicate | 13.0 | 13.0 | 13.0 | 13.0 | 13.0 |
| (CAS: 1344-09-8) | | | | | |
| Sodium metasilicate | 4.5 | 4.5 | 4.5 | 4.5 | 4.5 |
| (CAS: 6834-92-0) | | | | | |
| Ammonium bicarbonate | 13.33 | 13.33 | 13.33 | 13.33 | 13.33 |
| Behenyl behenate | 9.17 | — | — | — | — |
| (Compound 27) | | | | | |
| Dicaprylyl carbonate | — | 9.17 | — | — | — |
| (Compound 31) | | | | | |
| Isocetyl stearate | — | — | 9.17 | — | — |
| (Compound 33) | | | | | |
| Triethyl citrate | — | — | — | 9.17 | — |
| (Compound 37) | | | | | |
| Ethylhexyl stearate | — | — | — | — | 9.17 |
| (Compound 41) | | | | | |

TABLE 7

| Ingredients | C28 | C29 | C30 | C31 | C32 |
|---|---|---|---|---|---|
| Oxidizing cream | 60.0 | 60.0 | 60.0 | 60.0 | 60.0 |
| Blond Studio 30 Vol. | (i.e. 5.4 | (i.e. 5.4 | (i.e. 5.4 | (i.e. 5.4 | (i.e. 5.4 |
| L'Oréal Professionnel | of | of | of | of | of |
| (9% of H₂O₂) | H₂O₂) | H₂O₂) | H₂O₂) | H₂O₂) | H₂O₂) |
| Sodium silicate | 13.0 | 13.0 | 13.0 | 13.0 | 13.0 |
| (CAS: 1344-09-8) | | | | | |
| Sodium metasilicate | 4.5 | 4.5 | 4.5 | 4.5 | 4.5 |
| (CAS: 6834-92-0) | | | | | |
| Ammonium bicarbonate | 13.33 | 13.33 | 13.33 | 13.33 | 13.33 |
| Polyglyceryl-4 caprate | 9.17 | — | — | — | — |
| (Compound 53) | | | | | |
| Polyglyceryl-3 | — | 9.17 | — | — | — |
| methylglucose distearate | | | | | |
| (Compound 54) | | | | | |
| Polyglyceryl-2 stearate | — | — | 9.17 | — | — |
| (Compound 55) | | | | | |
| Polyglyceryl-2 | — | — | — | 9.17 | — |
| dipolyhydroxystearate | | | | | |
| (Compound 58) | | | | | |
| Polyglyceryl-6 dicaprate | — | — | — | — | 9.17 |
| (Compound 60) | | | | | |

TABLE 8

| Ingredients | C33 | C34 | C35 |
|---|---|---|---|
| Oxidizing cream | 60.0 | 60.0 | — |
| Blond Studio 30 Vol. | (i.e. 5.4 | (i.e. 5.4 | |
| L'Oréal Professionnel | of | of | |
| (9% of H₂O₂) | H₂O₂) | H₂O₂) | |
| Oxidizing cream | — | — | 60.0 |
| Blond Studio 40 Vol. | | | (i.e. 7.2 |
| L'Oreal Professionnel | | | of |
| (12% of H₂O₂) | | | H₂O₂) |
| Sodium silicate | 13.0 | 13.0 | 13.0 |
| (CAS: 1344-09-8) | | | |
| Sodium metasilicate | 4.5 | 4.5 | 4.5 |
| (CAS: 6834-92-0) | | | |
| Ammonium bicarbonate | 13.33 | 13.33 | 13.33 |
| Polyglyceryl-6 dioleate | 9.17 | — | — |
| (Compound 62) | | | |
| Dicaprylyl maleate | — | 9.17 | — |
| (Compound 63) | | | |
| Methyl glucose sesquistearate | — | — | 5.00 |
| (Glucate SS emulsifier | | | |
| marketed by Lubrizol) | | | |
| Water | — | — | 4.17 |

### Application Protocol

10 g of each of the compositions C13 to C35 are applied to 23 locks of 1 g of Caucasian HT4 hair on a hot plate maintained at a temperature of 33° C. The whole thing is covered with a cellophane film for 50 min.

The locks are subsequently rinsed, washed with a standard shampoo, rinsed again and then dried.

### Colorimetric Measurements

The results of the colorimetric measurements are summarized in the following table:

TABLE 9

| Composition | L* | b* |
|---|---|---|
| C13 | 49.09 | 22.86 |
| C14 | 42.78 | 21.03 |
| C15 | 45.6 | 23.02 |
| C16 | 50.22 | 24.68 |
| C17 | 49.05 | 23.96 |
| C18 | 48.69 | 23.1 |
| C19 | 46.81 | 22.93 |
| C20 | 49.31 | 23.02 |
| C21 | 49.01 | 23.24 |
| C22 | 49.31 | 24.27 |
| C23 | 48.39 | 24.47 |
| C24 | 47.01 | 24.38 |
| C25 | 46.93 | 23.35 |
| C26 | 49.46 | 24.25 |
| C27 | 47.27 | 23.74 |
| C28 | 47.69 | 23.66 |
| C29 | 49.11 | 23.37 |
| C30 | 50.29 | 24.5 |
| C31 | 52.08 | 24.1 |
| C32 | 45.35 | 23.35 |
| C33 | 48.98 | 23.79 |
| C34 | 48.09 | 23.46 |
| C35 | 46.72 | 23.87 |

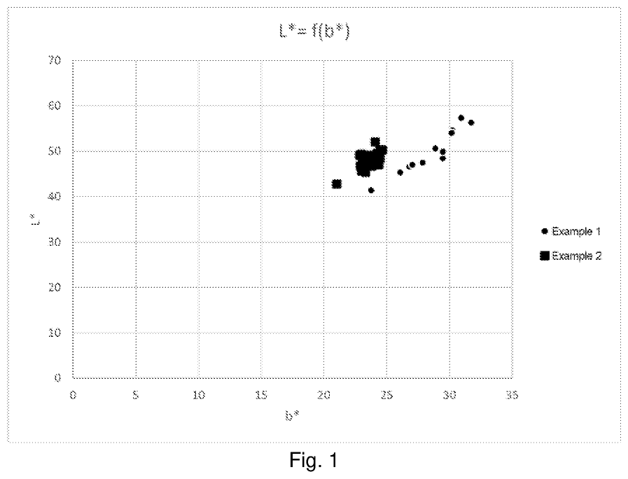

The results show that compositions according to the invention make it possible to obtain a good level of lightening characterized by relatively high values of L*. Furthermore, the colour shades obtained are characterized by values of b* which are lower, at an equivalent level of intensity L*, for the compositions according to the present invention than for the persulfate-based comparative compositions of Example 1, as illustrated in FIG. 1.

### Example 3

The following compositions D1 and D2 were prepared and then applied according to the application protocol described below:

TABLE 10

| Ingredients | D1 | D2 |
|---|---|---|
| Oxidizing cream | 60.0 | 60.0 |
| Blond Studio 30 Vol. | (i.e. 5.4 | (i.e. 5.4 |
| L'Oréal Professionnel | of | of |
| (9% of H₂O₂) | H₂O₂) | H₂O₂) |
| Sodium Silicate | 13.0 | 13.0 |
| (CAS: 1344-09-8) | | |
| Sodium Metasilicate | 4.5 | 4.5 |
| (CAS: 6834-92-0) | | |
| Ammonium Bicarbonate | 13.3 | 13.3 |
| Polyglyceryl-2 | 8.70 | 8.70 |
| Dipolyhydroxystearate | | |
| (Compound 58) | | |
| HC Blue 15 | 0.5 | — |
| Basic Red 51 | — | 0.5 |

### Application Protocol

10 g of each of the compositions D1 and D2 are applied to 2 locks of 1 g of dark Caucasian HT4 hair on a hot plate maintained at a temperature of 33° C. The whole thing is left in the open air for 50 min.

The locks are subsequently rinsed, washed with a standard shampoo, rinsed again and then dried.

### Results

TABLE 11

| Composition | Observed hair colour |
|---|---|
| D1 | Very light yellow-green |
| D2 | Red |

The compositions according to the invention make it possible to simultaneously bleach and dye dark hair in a single step and make it possible to obtain visible colours.

### Example 4

The following compositions E1 and E2 were prepared and then applied according to the application protocol described below:

TABLE 12

| Ingredients | E1 | E2 |
|---|---|---|
| Oxidizing cream | 60.0 | 60.0 |
| Blond Studio 30 Vol. | (i.e. 5.4 | (i.e. 5.4 |
| L'Oréal Professionnel | of | of |
| (9% of H₂O₂) | H₂O₂) | H₂O₂) |
| Sodium Silicate | 13.0 | 13.0 |
| (CAS: 1344-09-8) | | |
| Sodium Metasilicate | 4.5 | 4.5 |
| (CAS: 6834-92-0) | | |
| Ammonium Bicarbonate | 13.3 | 13.3 |
| Polyglyceryl-2 Dipolyhydroxystearate | 8.70 | 8.70 |
| (Compound 58) | | |
| Toluene-2,5-Diamine | 0.23 | — |
| 1-Hydroxyethyl 4,5-Diamino Pyrazole | — | 0.26 |
| Sulfate | | |
| 2,4-Diaminophenoxyethanol Hydrochloride | 0.27 | 0.24 |

### Application Protocol

10 g of each of the compositions E and E2 are applied to two locks of 1 g of dark Caucasian HT4 hair on a hot plate maintained at a temperature of 33° C. The whole thing is left in the open air for 50 min.

The lock is subsequently rinsed, washed with a standard shampoo, rinsed again and then dried.

### Results:

TABLE 13

| Composition | Observed hair colour |
|---|---|
| E1 | Yellowish brown |
| E2 | Reddish brown |

The compositions according to the invention make it possible to simultaneously bleach and dye dark hair in a single step and make it possible to obtain visible colours.

1-29. (canceled)

30. A composition for lightening keratin fibers comprising:
i) at least one chemical oxidizing agent chosen from hydrogen peroxide, a hydrogen peroxide-generating system other than a peroxygenated salt, or mixtures thereof;
ii) at least one compound chosen from a carbonate, a carbonate-generating system, a bicarbonate, a bicarbonate-generating system, or mixtures thereof;
iii) at least one silicate;
iv) at least one compound chosen from an ester, an amide, an imine, or mixtures thereof;
wherein the total amount of silicate ranges from 1% to 40% by weight, relative to the total weight of the composition; and
wherein the composition comprises persulfates in a total amount of less than 5% by weight, relative to the total weight of the composition.

31. The composition of claim 30, wherein the chemical oxidizing agent is hydrogen peroxide.

32. The composition of claim 30, wherein the total amount of chemical oxidizing agent ranges from 1% to 12% by weight, relative to the total weight of the composition.

33. The composition of claim 30, wherein the total amount of compound ii) ranges from 0.01% to 20% by weight, relative to the total weight of the composition.

34. The composition of claim 30, wherein the at least one compound ii) is chosen from a carbonate, a carbonate-generating system, or mixtures thereof.

35. The composition of claim 34, wherein the total amount of carbonate and/or carbonate-generating system ranges from 0.01% to 20% by weight, relative to the total weight of the composition.

36. The composition of claim 35, wherein the at least one compound ii) is a carbonate chosen from:
an alkali metal carbonate;
an alkaline earth metal carbonate;
a lanthanide carbonate;
a transition metal carbonate;
a bismuth carbonate;
a cadmium carbonate;
a thallium carbonate;
a zinc carbonate;
a compound of formula (N⁺R¹R²R³R⁴)₂, —CO₃²⁻ wherein R¹, R², R³ and R⁴ are independently chosen from a hydrogen atom or a (C₁-C₄)alkyl group optionally substituted by a hydroxyl group;
a guanidine carbonate; or
mixtures thereof.

37. The composition of claim 30, wherein the at least one compound ii) is chosen from a bicarbonate, a bicarbonate-generating system, or mixtures thereof.

38. The composition of claim 37, wherein the total amount of bicarbonate and/or bicarbonate-generating system ranges from 0.01% to 20% by weight, relative to the total weight of the composition.

39. The composition of claim 37, wherein the at least one compound ii) is a bicarbonate chosen from:
an alkali metal bicarbonate;
an alkaline earth metal bicarbonate;
a compound of formula N⁺R¹R²R³R⁴, —HCO₃⁻ wherein R¹, R², R³ and R⁴ are independently chosen from a hydrogen atom or a (C₁-C₄)alkyl group optionally substituted by a hydroxyl group;
an aminoguanidine bicarbonate; or
mixtures thereof.

40. The composition of claim 30, wherein the at least one silicate is chosen from an alkali metal silicate, an alkaline earth metal silicate, an aluminium silicate, a trimethylammonium silicate, or mixtures thereof.

41. The composition of claim 30, wherein the total amount of silicate ranges from 1% to 40% by weight, relative to the total weight of the composition.

42. The composition of claim 30, wherein the weight ratio of the total amount of bicarbonate and/or bicarbonate-generating system to the total amount of silicate ranges from 1:50 to 15:2.

43. The composition of claim 30, wherein the weight ratio of the total amount of bicarbonate and/or bicarbonate-generating system to the total amount of chemical oxidizing agent ranges from 11:100 to 5:1.

44. The composition of claim 30, wherein the weight ratio of the total amount of carbonate and/or carbonate-generating system to the total amount of bicarbonate and/or bicarbonate-generating system ranges from 3:50 to 20:1.

45. The composition of claim 30, wherein the at least one compound iv) is chosen from a compound of formula (I), a salt thereof, an optical isomer thereof, a geometric isomer thereof, a tautomer thereof, a solvate thereof, or mixtures thereof: 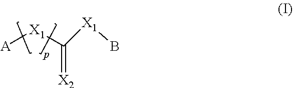
wherein in formula (I):
A represents a saturated or unsaturated, linear or branched hydrocarbon group comprising from 1 to 30 carbon atoms, optionally interrupted by one or more nonadjacent oxygen atoms and/or by one or more identical or different groups chosen from: —CO—, —OCO—, —COO—, and/or optionally substituted by one or more identical or different groups chosen from: a hydroxyl group (—OH), a group —COOAk1, a (hetero)cyclic group optionally substituted by one or more hydroxyl groups (—OH);
X₁ independently represents an oxygen atom or a group —NR—;
X₂ represents an oxygen atom or a group —NR—;
p=0 or 1;
B represents a saturated or unsaturated, linear or branched or cyclic hydrocarbon group, comprising from 1 to 30 carbon atoms, optionally substituted by one or more identical or different groups chosen from:
a (hetero)cyclic group optionally substituted by one or more identical or different groups chosen from: hydroxyl (—OH), —OR″, —COOA′, —OCOA′, (poly)(hydroxy)alkyl;
a group —OR′;
a group —COOA′;
a group —OCOA′;
and/or optionally interrupted by one or more nonadjacent oxygen atoms and/or by one or more identical or different groups chosen from: —OCO—, —COO—;
A′ represents a saturated or unsaturated, linear or branched hydrocarbon group comprising from 1 to 30 carbon atoms, optionally interrupted by one or more nonadjacent oxygen atoms and/or by one or more identical or different groups chosen from: —CO—, —OCO—, —COO—, and/or optionally substituted by one or more identical or different groups chosen from: hydroxyl (—OH), —COOAk1;
R represents a hydrogen atom or an alkyl group;
R′ represents a hydrogen atom or an alkyl group optionally substituted by one or more identical or different groups chosen from: hydroxyl (—OH) or —OCOA′;
R″ represents:
an alkyl group optionally substituted by one or more hydroxyl groups (—OH); or
a (hetero)cyclic group optionally substituted by one or more identical or different groups chosen from: hydroxyl (—OH), (poly)(hydroxy)alkyl;
Ak1 represents an alkyl group optionally substituted by —COOAk2;
Ak2 represents an alkyl group;
wherein when X₁ represents a group —NR—, A and B represent a hydrogen atom.

46. The composition of claim 30, wherein the total amount of compound iv) ranges from 0.01% to 50% by weight, relative to the total weight of the composition.

47. The composition of claim 30, wherein the pH of the composition ranges from 8 to 11.

48. The composition of claim 30, wherein the composition further comprises at least one coloring agent chosen from a direct dye, an oxidation dye, or mixtures thereof, wherein the total amount of coloring agent is less than 0.1% by weight, relative to the total weight of the composition.

49. A method for lightening keratin fibers, comprising:
(i) mixing a composition (A) with a composition (B) and optionally a composition (C), wherein composition (A) comprises:
at least one chemical oxidizing agent chosen from hydrogen peroxide, a hydrogen peroxide-generating system other than peroxygenated salts, or mixtures thereof; and
wherein composition (B) comprises:
at least one compound chosen from a carbonate, a carbonate-generating system, a bicarbonate, a bicarbonate-generating system, or mixtures thereof;
at least one silicate;
optionally at least one coloring agent chosen from a direct dye, an oxidation dye, or mixtures thereof; and
optionally at least one compound chosen from an ester, an amide, an imine, or mixtures thereof; and
wherein composition (C) comprises:
at least one coloring agent chosen from a direct dye, an oxidation dye, or mixtures thereof; and
optionally at least one compound chosen from an ester, an amide, an imine, or mixtures thereof;
wherein composition (B), or at least one of composition (B) or composition (C) when composition (C) is present, comprises the at least one compound chosen from an ester, an amide, an imine, or mixtures thereof; and
(ii) applying the mixture to the keratin fibers.
